(19) United States
(12) Patent Application Publication
(10) Pub. No.: US 2025/0075258 A1
(43) Pub. Date: Mar. 6, 2025

(54) METHODS OF DETECTING SJOGREN'S SYNDROME USING SALIVARY EXOSOMES

(71) Applicants: Exosome Diagnostics, Inc., Waltham, MA (US); Trustees of Tufts College, Medford, MA (US)

(72) Inventors: Sudipto Kumar CHAKRABORTTY, Waltham, MA (US); Steven LANG, Waltham, MA (US); Yevgenia Leona Khodor TOLAN, Waltham, MA (US); Martina RAUSCHER, Waltham, MA (US); Vasisht TADIGOTLA, Waltham, MA (US); Wei YU, Waltham, MA (US); Athena S. PAPAS, Medford, MA (US); Johan Karl Olov SKOG, Waltham, MA (US); Brian C. HAYNES, Austin, TX (US); Shuran XING, Waltham, MA (US)

(21) Appl. No.: 18/579,636

(22) PCT Filed: Jul. 18, 2022
(86) PCT No.: PCT/US2022/037469

§ 371 Date: Jan. 16, 2024

Related U.S. Application Data
(60) Provisional application No. 63/222,823, filed on Jul. 16, 2021.

(51) Int. Cl. C12Q 1/6832; C12Q 1/6869

(52) U.S. Cl. CPC C12Q 1/6832; C12Q 1/6869; C12Q 2600/158

(57) ABSTRACT

The present disclosure is directed to methods of using salivary exosomes to detect and treat Sjögren's syndrome in a subject.

### RELATED APPLICATIONS

This application claims priority to, and the benefit of, U.S. Provisional Application No. 63/222,823, filed Jul. 16, 2021, the contents of which are incorporated herein by reference in their entireties.

### BACKGROUND OF THE INVENTION

Sjögren's syndrome (SS) is a systemic autoimmune disease in which inflammation progressively damages the moisture-producing glands such as the salivary glands and tear glands. An estimated four million Americans are thought to suffer from the disease with estimated 2.5 million undiagnosed, 90% of which are women with an average age of 40. The symptoms of SS overlap with other health conditions and co-morbidities, including other inflammatory diseases and disorders, other auto-immune diseases, allergies, drug side effects and menopause. Moreover, the diagnostic criteria have changed multiple times because autoantibodies are not present in all patients and salivary gland biopsy material can be difficult to obtain and pathologists may not correctly apply standard diagnostic criteria, making SS difficult to diagnose. In fact, the average time to diagnosis is currently three years. While several biomarker diagnostic tests have been developed for Sjögren's syndrome, the gold standard remains the biopsy of salivary glands, which is an invasive, expensive, and time-consuming procedure with a potential side effect of permanent lip numbness. Thus, there is a need in the art for improved methods for diagnosing Sjögren's syndrome.

### SUMMARY OF THE INVENTION

The present disclosure provides methods of identifying the presence or absence of Sjögren's syndrome in a subject, the methods comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; and b) identifying the presence of Sjögren's syndrome in the subject based on the expression level of the at least one biomarker.

In some aspects, identifying the presence of Sjögren's syndrome in the subject based on the expression level of the at least one biomarker can comprise: i) inputting the expression levels from step (a) into an algorithm to generate a score; ii) comparing the score to a predetermined cutoff value; and iii) determining the presence or absence of Sjögren's syndrome in the subject based on comparison between the score and the predetermined cutoff value.

In some aspects, an algorithm can be the product of a feature selection wrapper algorithm, a machine learning algorithm, a trained classifier built from at least one predictive classification algorithm or any combination thereof. In some aspects, a predictive classification algorithm, a feature selection wrapper algorithm, and/or a machine learning algorithm can comprise XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

In some aspects, an algorithm can the product of a trained classifier is trained to identify Sjögren's syndrome in a subject using: i) the expression level of the at least one biomarker in at least one biological sample from at least one subject who does not have Sjögren's syndrome; and ii) the expression levels of the at least one biomarker in at least one biological sample from at least one subject who has Sjögren's syndrome.

In some aspects of the preceding methods, the at least one biomarker can be at least one of IFIT3, OAS1, OAS2, IFIT1, TMEM60 and CPNE2, wherein the presence of Sjögren's syndrome in the subject is identified when the expression level of the at least one biomarker is greater than or equal to a predetermined cutoff value; and/or

In some aspects of the preceding methods, the at least one biomarker can ZNF395, wherein the presence of Sjögren's syndrome in the subject is identified when the expression level of the at least one biomarker is less than or equal to a predetermined cutoff value

In some aspects of the preceding methods, step (a) can comprise determining the expression level of: a) at least two of the seven biomarkers; b) at least three of the seven biomarkers; c) at least four of the seven biomarkers; d) at least five of the seven biomarkers; e) at least six of the seven biomarkers; or f) each of the seven biomarkers.

In some aspects, the preceding methods can further comprise, prior to step (a): i) isolating a plurality of microvesicles from the saliva sample from the subject; and ii) extracting at least one microvesicular RNA from the plurality of isolated microvesicles.

In some aspects, the at least one microvesicle can be isolated from the saliva sample by contacting the saliva sample with at least one affinity agent that binds to at least one surface marker present on the surface the at least one microvesicle.

In some aspects of the preceding methods, step (a) further comprises: (i) determining the expression level of at least one reference biomarker; and (ii) normalizing the expression level of the at least one biomarker to the expression level of the at least one reference biomarker.

In some aspects, inputting expression levels from into an algorithm to generate a score comprises inputting the normalized expression levels into an algorithm to generate a score.

In some aspects, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

In some aspects, determining the expression level of a biomarker can comprise sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

In some aspects, a predetermined cutoff value can have a negative predictive value of at least about 80%, or at least about 85%, or at least about 90%, or at least about 95%, or at least about 99%, or at least about 99.9%.

In some aspects, a predetermined cutoff value can have a positive predictive value of at least about 80%, or at least about 85%, or at least about 90%, or at least about 95%, or at least about 99%, or at least about 99.9%.

In some aspects, a predetermined cutoff value can have a sensitivity of at least about 80%, or at least about 85%, or at least about 90%, or at least about 95%, or at least about 99%, or at least about 99.9%.

In some aspects, a predetermined cutoff value can have a specificity of at least about 80%, or at least about 85%, or at least about 90%, or at least about 95%, or at least about 99%, or at least about 99.9%.

In some aspects, a predetermined cutoff value can be calculated using at least one receiver operating characteristic (ROC) curve.

In some aspects, measuring expression levels can further comprise selectively enriching for at least one biomarker. In some aspects, an at least one biomarker can be selectively enriched by hybrid-capture. In some aspects, hybrid-capture can substantially enrich nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome. In some aspects, hybrid-capture can result in a significant depletion in microbial nucleic acids

In some aspects, the preceding methods can further comprise administering at least one treatment to a subject identified as having Sjögren's syndrome. In some aspects, an at least one treatment can comprise: i) administering at least one therapeutically effective amount of an cevimeline (Evoxac®) pilocarpine (Salagen®), a supersaturated calcium phosphate rinse (e.g. NeutraSal®), cyclosporine (including ophthalmic emulsions, e.g. Restasis®) and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof; ii) surgery, preferably wherein the surgery comprises sealing the tear ducts of the subject; or iii) a combination thereof.

In some aspects, a saliva sample can be collected at the subject's home through the use of a sample home-collection device.

The present disclosure provides a method of identifying the presence or absence of Sjögren's syndrome in a subject, the method comprising: a) determining the expression level of at least one biomarker in microvesicular RNA isolated from a saliva sample from the subject; b) comparing the expression level of the at least one biomarker to a corresponding predetermined cutoff value; c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

The present disclosure provides a method of monitoring a Sjögren's syndrome treatment in a subject, wherein the subject has been administered the Sjögren's syndrome treatment, the method comprising: a) determining the expression level of at least one biomarker in microvesicular RNA isolated from a saliva sample from the subject; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one biomarker in microvesicular RNA isolated from a saliva sample from the subject, b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of identifying the presence or absence of Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method of monitoring a Sjögren's syndrome treatment in a subject, wherein the subject has been administered the Sjögren's syndrome treatment, the method comprising: a) determining the expression level of at least one biomarker in microvesicular RNA isolated from a saliva sample from the subject; b) inputting the expression levels from step (a) into an algorithm to generate a score; c) comparing the score to a predetermined cutoff value; d) determining whether the patient is responding to the Sjögren's syndrome treatment based on whether the score is greater than, equal to, or less than the predetermined cutoff value.

In some aspects the preceding methods can further comprise, prior to step (a): i) isolating a plurality of microvesicles from the saliva sample from the subject; and ii) extracting at least one microvesicular RNA from the plurality of isolated microvesicles. Isolating a plurality of microvesicles from a biological sample from the subject can comprise a processing step to remove cells, cellular debris or a combination of cells and cellular debris. A processing step can comprise filtering the sample, centrifuging the sample, or a combination of filtering the sample and centrifuging the sample. Centrifuging can comprise centrifuging at about 2000×g. Filtering can comprise filtering the sample through a filter with a pore size of about 0.8 microns.

In some aspects, isolating a plurality of microvesicles comprises ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the preceding methods, the at least one microvesicle is isolated from the saliva sample by contacting the saliva sample with at least one affinity agent that binds to at least one surface marker present on the surface the at least one microvesicle.

In some aspects, the preceding methods, step (a) can further comprise: (i) determining the expression level of at least one reference biomarker; (ii) normalizing the expression level of the at least one biomarker to the expression level of the at least one reference biomarker.

In some aspects, inputting the expression levels from step (a) into an algorithm to generate a score can comprise inputting the normalized expression levels from step (a) into an algorithm to generate a score.

In some aspects, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof. In some aspects of the preceding methods, wherein determining the expression level of a biomarker comprises sequencing, next-generation sequencing (NGS), high-throughput sequencing or any combination thereof, at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

In some aspects, a predetermined cutoff value can have a negative predictive value of at least about 80%, or at least about 85%, or at least about 90%, or at least about 95%, or at least about 99%, or at least about 99.9%. In some aspects, a predetermined cutoff value can have a positive predictive value of at least about 80%, or at least about 85%, or at least about 90%, or at least about 95%, or at least about 99%, or at least about 99.9%. In some aspects, a predetermined cutoff value can have a sensitivity of at least about 80%, or at least about 85%, or at least about 90%, or at least about 95%, or at least about 99%, or at least about 99.9%.

In some aspects, a predetermined cutoff value can have a specificity of at least about 80%, or at least about 85%, or at least about 90%, or at least about 95%, or at least about 99%, or at least about 99.9%.

In some aspects, an algorithm is the product of a feature selection wrapper algorithm. In some aspects, an algorithm is the product of a machine learning algorithm. In some aspects, an algorithm is the product of a trained classifier built from at least one predictive classification algorithm. A predictive classification algorithm, the feature selection wrapper algorithm, and/or the machine learning algorithm can comprise XGBoost (XGB), random forest (RF), Lasso and Elastic-Net Regularized Generalized Linear Models (glmnet), cforest, classification and regression tree (CART), treebag, k nearest-neighbor (knn), neural network (nnet), support vector machine-radial (SVM-radial), support vector machine-linear (SVM-linear), naïve bayes (NB), multilayer perceptron (mlp) or any combination thereof. An algorithm can be the product of a logistic regression model. A logistic regression model can comprise a LASSO regularization.

In some aspects, a predetermined cutoff value can be calculated using at least one receiver operating characteristic (ROC) curve.

In some aspects of the preceding methods, measuring expression levels in step (a) further comprises selectively enriching for at least one biomarker. In some aspects of the preceding methods, the at least one biomarker is selectively enriched by hybrid-capture. In some aspects of the preceding methods, hybrid-capture substantially enriches nucleic acid transcripts that correspond to the human transcriptome. In some aspects of the preceding methods, hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome. In some aspects of the preceding methods, hybrid-capture results in a significant depletion in microbial nucleic acids.

The preceding methods can further comprise administering at least one treatment to a subject identified as having Sjögren's syndrome. The at least one treatment can comprise administering at least one therapeutically effective amount of an cevimeline (Evoxac®) pilocarpine (Salagen®), a supersaturated calcium phosphate rinse (e.g. NeutraSal®), cyclosporine (including ophthalmic emulsions, e.g. Restasis® and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof. A treatment can comprise surgery. Surgery can comprise sealing the tear ducts of the subject.

In some aspects, a saliva sample can be collected at the subject's home through the use of a sample home-collection device.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising.” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and further features will be more clearly appreciated from the following detailed description when taken in conjunction with the accompanying drawings.

FIG. 1 is a series of charts showing the distribution of unmapped, intergenic, intronic, transcriptome and other genomic region sequencing reads in the analysis of microvesicular RNA isolated from salivary microvesicles from subjects with Sjögren's syndrome and healthy subjects.

FIG. 2 is a series of charts showing the distribution of biotypes in the sequencing reads in the analysis of microvesicular RNA isolated from salivary microvesicles from subjects with Sjögren's syndrome and healthy subjects.

FIG. 3 is a series of charts showing on-target, enriched faction and mapped sequencing reads in the sequencing analysis of microvesicular RNA isolated from salivary microvesicles from subjects with Sjögren's syndrome and healthy subjects.

FIG. 4 is a chart showing the number of genes detected and the number of genes detected with a coverage of greater than or equal to 80% in the sequencing analysis of microvesicular RNA isolated from salivary microvesicles from subjects with Sjögren's syndrome and healthy subjects.

FIG. 5 is a series of charts showing Principle Component Analysis (PCA) of the expression of all genes (left panel) and differentially expressed genes (right panel) in sequencing analysis of microvesicular RNA isolated from salivary microvesicles from subjects with Sjögren's syndrome and healthy subjects

FIG. 6 is a chart showing PCA analysis of the expression of ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9 in microvesicular RNA isolated from salivary microvesicles from subjects with Sjögren's syndrome and healthy subjects.

FIG. 7 shows a graph depicting certain groups of genes that were enriched in either Sjögren's syndrome samples or healthy samples.

FIG. 8 is a series of charts showing the distribution of unmapped, intergenic, intronic, transcriptome and other genomic region sequencing reads in the analysis of microvesicular RNA isolated from salivary microvesicles from subjects with Sjögren's syndrome and healthy subjects.

FIG. 9 is a series of charts showing the distribution of biotypes in the sequencing reads in the analysis of microvesicular RNA isolated from salivary microvesicles from subjects with Sjögren's syndrome and healthy subjects.

FIG. 10 is a graph showing leave-one out cross-validation analysis of a gene signature for the identification of Sjögren's syndrome, the gene signature comprising IFIT3, OAS1, OAS2, IFIT1, TMEM60, ZNF395 and CPNE2.

FIG. 11 is a graph showing the expression levels of IFIT3, OAS1, OAS2, IFIT1, TMEM60, ZNF395 and CPNE2 in microvesicular RNA collected from saliva samples from subjects with Sjögren's syndrome (left side) and from healthy subjects (right side).

### DETAILED DESCRIPTION OF THE INVENTION

The present disclosure provides methods of identifying and treating Sjögren's syndrome in a subject in need thereof.

Sjögren's syndrome (SS) is a systemic autoimmune disease in which inflammation progressively damages the moisture producing glands such as the salivary glands and the tear glands. Symptoms can include dry, irritated and red eyes, dry mouth and difficulty swallowing. Four million Americans are estimated to be suffering from the disease, 90% of which are women with an average age of 40.

Overlapping symptoms with other health conditions and co-morbidities make SS particularly difficult to diagnose, with average time to diagnosis of 3 years. Currently, diagnosis of SS is performed by either: a) measuring levels of SS-A (Ro) protein in a biological sample from a subject (about 70% of subjects with SS test positive for SS-A); b) measuring levels of SS-B (La) protein in a biological sample from a subject (about 40% of subjects with SS test positive for SS-B); c) measuring levels of anti-nuclear antibody (ANA) in a biological sample from a subject (about 70% of subjects with SS test positive for ANA) but ANA is also a marker for other autoimmune diseases such as systemic lupus erythematosus; d) measuring levels of rheumatoid factor (RF) in a biological sample from a subject (about 60%-70% of subjects with SS test positive for RF, but RF is also a marker for other rheumatic diseases such as rheumatoid arthritis and systemic lupus erythematosis); or e) performing a salivary gland biopsy (typically from the lower lip) to confirm inflammatory cell infiltration of the minor salivary glands. Of all the current diagnostic methods, salivary gland biopsy is considered the “gold standard”. However, biopsy is an invasive, expensive, and time-consuming procedure. Moreover, biopsy can could potentially lead to permanent lip numbness. Thus, there is a clear need in the art for a less-invasive, inexpensive and easy-to-administer diagnostic test for SS.

Analysis of salivary exosomes has primarily focused on small RNAs and has been limited due to the large contribution of sequencing reads from the oral microbiome. In fact, previous studies that analyzed microvesicular RNA extracted from salivary microvesicles found that ˜60-95% of sequencing reads mapped to exogenous (i.e. microbial) genomes and transcriptomes. The methods of the present disclosure overcome these previous limitations and unexpectedly allows for the analysis of mRNAs and long intervening/intergenic noncoding RNAs (lincRNAs) in nucleic acids extracted from salivary microvesicles. For the purpose of the present disclosure microvesicular RNA, interchangeable with extracellular RNA (exRNA) or cell-free RNA, describes RNA species present outside of the cells in which they were transcribed. Carried within extracellular vesicles, lipoproteins, and protein complexes, exRNAs are protected from ubiquitous RNA-degrading enzymes. exRNAs may be found in the environment or, in multicellular organisms, within the tissues or biological fluids such as venous blood, saliva, breast milk, vaginal fluid, urine, semen, and menstrual blood.

The present disclosure provides a method of identifying the presence or absence of Sjögren's syndrome in a subject, the method comprising analyzing the expression level of at least one biomarker in microvesicular RNA isolated from a saliva sample from the subject.

The present disclosure provides a method of treating Sjögren's syndrome in a subject, the method comprising analyzing the expression level of at least one biomarker in microvesicular RNA isolated from a saliva sample from the subject and administering at least one treatment to a subject identified as having Sjögren's syndrome based on the analysis of the microvesicular RNA.

The present disclosure provides a method of monitoring a Sjögren's syndrome treatment in a subject that has been administered the Sjögren's syndrome treatment, the method comprising analyzing the expression level of at least one biomarker in microvesicular RNA isolated from a saliva sample from the subject and determining whether the patient is responding to the Sjögren's syndrome treatment based on the analysis of the microvesicular RNA.

The present disclosure provides a method of identifying the presence or absence of Sjögren's syndrome in a subject, the method comprising analyzing the expression level of at least one biomarker in microvesicular RNA and cell-free DNA (cfDNA) isolated from a saliva sample from the subject.

The present disclosure provides a method of treating Sjögren's syndrome in a subject, the method comprising analyzing the expression level of at least one biomarker in microvesicular RNA and cell-free DNA isolated from a saliva sample from the subject and administering at least one treatment to a subject identified as having Sjögren's syndrome based on the analysis of the microvesicular RNA and cell-free DNA.

The present disclosure provides a method of monitoring a Sjögren's syndrome treatment in a subject that has been administered the Sjögren's syndrome treatment, method comprising analyzing the expression level of at least one biomarker in microvesicular RNA and cell-free DNA isolated from a saliva sample from the subject and determining whether the subject is responding to the Sjögren's syndrome treatment based on the analysis of the microvesicular RNA and cell-free DNA.

The present disclosure provides a method of identifying the presence or absence of Sjögren's syndrome in a subject, the method comprising: a) determining the expression level of at least one biomarker in microvesicular RNA isolated from a saliva sample from the subject; and b) identifying the presence or absence of Sjögren's syndrome based on the expression level of the at least one biomarker. In some aspects, identifying the presence or absence of Sjögren's syndrome based on the expression level of the at least one biomarker can comprise comparing the one or more expression levels to corresponding predetermined cutoff value and determining the presence or absence of Sjögren's syndrome in the subject based on the relationship between the one or more expression levels and the corresponding predetermined cutoff values (e.g. greater than, less than, or equal to).

The present disclosure provides a method of monitoring a Sjögren's syndrome treatment in a subject that has been administered the Sjögren's syndrome treatment, the method comprising: a) determining the expression level of at least one biomarker in microvesicular RNA isolated from a saliva sample from the subject; and b) determining whether the subject is responding to the Sjögren's syndrome treatment based on the expression level of the at least one biomarker. In some aspects, determining whether the subject is responding to the Sjögren's syndrome treatment based on the expression level of the at least on biomarker can comprise comparing the one or more expression levels to predetermined cutoff values and determining if the subject is responding based on the relationship between the one or more expression levels and the corresponding predetermined cutoff values (e.g. greater than, less than, or equal to).

The present disclosure provides a method of treating Sjögren's syndrome in a subject, the method comprising: a) determining the expression level of at least one biomarker in microvesicular RNA isolated from a saliva sample from the subject; and b) administering at least one treatment to the subject based on the expression level of the at least one biomarker. In some aspects, administering at least one treatment to the subject based on the expression level of the at least one biomarker can further comprise comparing the one or more expression levels to corresponding predetermined cutoff values and determining if the treatment is needed based on the relationship between the one or more expression levels and the corresponding predetermined cutoff values (e.g. greater than, less than, or equal to).

The present disclosure provides a method of identifying the presence or absence of Sjögren's syndrome in a subject, the method comprising: a) determining the expression level of at least one biomarker in microvesicular RNA isolated from a saliva sample from the subject; b) comparing the expression level of the at least one biomarker to a corresponding predetermined cutoff value; c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

The present disclosure provides a method of treating Sjögren's syndrome in a subject, the method comprising: a) determining the expression level of at least one biomarker in microvesicular RNA isolated from a saliva sample from the subject; b) comparing the expression level of the at least one biomarker to a corresponding predetermined cutoff value; c) administering at least one treatment to the subject when the expression level of the at least one biomarker is greater than or equal to its corresponding predetermined cutoff value.

The present disclosure provides a method of identifying the presence or absence of Sjögren's syndrome in a subject, the method comprising: a) determining the expression level of at least one biomarker in microvesicular RNA isolated from a saliva sample from the subject; b) comparing the expression level of the at least one biomarker to a corresponding predetermined cutoff value; c) identifying the presence of Sjögren's syndrome in the subject when the expression level of the at least one biomarker is less than or equal to its corresponding predetermined cutoff value or identifying the absence of Sjögren's syndrome in the subject when the expression level of the at least one biomarker is greater than its corresponding predetermined cutoff value.

The present disclosure provides a method of treating Sjögren's syndrome in a subject, the method comprising: a) determining the expression level of at least one biomarker in microvesicular RNA isolated from a saliva sample from the subject; b) comparing the expression level of the at least one biomarker to a corresponding predetermined cutoff value; c) administering at least one treatment to the subject when the expression level of the at least one biomarker is less than or equal to its corresponding predetermined cutoff value.

The present disclosure provides a method of monitoring a Sjögren's syndrome treatment in a subject that has been administered the Sjögren's syndrome treatment, the method comprising: a) determining the expression level of at least one biomarker in microvesicular RNA isolated from a saliva sample from the subject; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one of five biomarkers in microvesicular RNA isolated from a saliva sample from the subject, wherein the five biomarkers comprise OAS1, OAS2, IRF5, IFIT1, and IFIT3, and b) identifying the presence of Sjögren's syndrome in the subject based on the expression level of the at least one biomarker.

The present disclosure provides a method of treating Sjögren's syndrome in a subject, the method comprising: a) determining the expression level of at least one of five biomarkers in microvesicular RNA isolated from a saliva sample from the subject, wherein the five biomarkers comprise OAS1, OAS2, IRF5, IFIT1, and IFIT3; and b) administering at least one treatment to the subject based on the expression level of the at least one biomarker.

The present disclosure provides a method of monitoring a Sjögren's syndrome treatment in a subject that has been administered the Sjögren's syndrome treatment, the method comprising: determining the expression level of at least one of five biomarkers in microvesicular RNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one of five biomarkers in microvesicular RNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

The present disclosure provides a method of treating Sjögren's syndrome in a subject, the method comprising: a) determining the expression level of at least one of five biomarkers in microvesicular RNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) administering at least one treatment to the subject when the expression level of the at least one biomarker is greater than or equal to its corresponding predetermined cutoff value.

The present disclosure provides a method of monitoring a Sjögren's syndrome treatment in a subject that has been administered the Sjögren's syndrome treatment, the method comprising: determining the expression level of at least one of five biomarkers in microvesicular RNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

In some aspects of the preceding methods, step (a) can comprise determining the expression level of at least two of the five biomarkers, or at least three of the five biomarkers, or at least four of the five biomarkers, or each of the five biomarkers.

The present disclosure provides a method of identifying the presence or absence of Sjögren's syndrome in a subject, the method comprising: a) determining the expression level of at least one biomarker in microvesicular RNA and cell-free DNA isolated from a saliva sample from the subject; and b) identifying the presence or absence of Sjögren's syndrome based on the expression level of the at least one biomarker. In some aspects, identifying the presence or absence of Sjögren's syndrome based on the expression level of the at least one biomarker can comprise comparing the one or more expression levels to corresponding predetermined cutoff value and determining the presence or absence of Sjögren's syndrome in the subject based on the relationship between the one or more expression levels and the corresponding predetermined cutoff values (e.g. greater than, less than, or equal to).

The present disclosure provides a method of monitoring a Sjögren's syndrome treatment in a subject that has been administered the Sjögren's syndrome treatment, the method comprising: a) determining the expression level of at least one biomarker in microvesicular RNA and cell-free DNA isolated from a saliva sample from the subject; and b) determining whether the subject is responding to the Sjögren's syndrome treatment based on the expression level of the at least one biomarker. In some aspects, determining whether the subject is responding to the Sjögren's syndrome treatment based on the expression level of the at least on biomarker can comprise comparing the one or more expression levels to predetermined cutoff values and determining if the subject is responding based on the relationship between the one or more expression levels and the corresponding predetermined cutoff values (e.g. greater than, less than, or equal to).

The present disclosure provides a method of treating Sjögren's syndrome in a subject, the method comprising: a) determining the expression level of at least one biomarker in microvesicular RNA and cell-free DNA isolated from a saliva sample from the subject; and b) administering at least one treatment to the subject based on the expression level of the at least one biomarker. In some aspects, administering at least one treatment to the subject based on the expression level of the at least one biomarker can further comprise comparing the one or more expression levels to corresponding predetermined cutoff values and determining if the treatment is needed based on the relationship between the one or more expression levels and the corresponding predetermined cutoff values (e.g. greater than, less than, or equal to).

The present disclosure provides a method of identifying the presence or absence of Sjögren's syndrome in a subject, the method comprising: a) determining the expression level of at least one biomarker in microvesicular RNA and cell-free DNA isolated from a saliva sample from the subject; b) comparing the expression level of the at least one biomarker to a corresponding predetermined cutoff value; c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

The present disclosure provides a method of treating Sjögren's syndrome in a subject, the method comprising: a) determining the expression level of at least one biomarker in microvesicular RNA and cell-free DNA isolated from a saliva sample from the subject; b) comparing the expression level of the at least one biomarker to a corresponding predetermined cutoff value, c) administering at least one treatment to the subject when the expression level of the at least one biomarker is greater than or equal to its corresponding predetermined cutoff value.

The present disclosure provides a method of identifying the presence or absence of Sjögren's syndrome in a subject, the method comprising: a) determining the expression level of at least one biomarker in microvesicular RNA and cell-free DNA isolated from a saliva sample from the subject; b) comparing the expression level of the at least one biomarker to a corresponding predetermined cutoff value; c) identifying the presence of Sjögren's syndrome in the subject when the expression level of the at least one biomarker is less than or equal to its corresponding predetermined cutoff value or identifying the absence of Sjögren's syndrome in the subject when the expression level of the at least one biomarker is greater than its corresponding predetermined cutoff value.

The present disclosure provides a method of treating Sjögren's syndrome in a subject, the method comprising: a) determining the expression level of at least one biomarker in microvesicular RNA and cell-free DNA isolated from a saliva sample from the subject; b) comparing the expression level of the at least one biomarker to a corresponding predetermined cutoff value; c) administering at least one treatment to the subject when the expression level of the at least one biomarker is less than or equal to its corresponding predetermined cutoff value.

The present disclosure provides a method of monitoring a Sjögren's syndrome treatment in a subject that has been administered the Sjögren's syndrome treatment, the method comprising: a) determining the expression level of at least one biomarker in microvesicular RNA and cell-free DNA isolated from a saliva sample from the subject; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one of five biomarkers in microvesicular RNA and cell-free DNA isolated from a saliva sample from the subject, wherein the five biomarkers comprise OAS1, OAS2, IRF5, IFIT1, and IFIT3; and b) identifying the presence of Sjögren's syndrome in the subject based on the expression level of the at least one biomarker.

The present disclosure provides a method of treating Sjögren's syndrome in a subject, the method comprising: a) determining the expression level of at least one of five biomarkers in microvesicular RNA and cell-free DNA isolated from a saliva sample from the subject, wherein the five biomarkers comprise OAS1, OAS2, IRF5, IFIT1, and IFIT3; and b) administering at least one treatment to the subject based on the expression level of the at least one biomarker.

The present disclosure provides a method of monitoring a Sjögren's syndrome treatment in a subject that has been administered the Sjögren's syndrome treatment, the method comprising: determining the expression level of at least one of five biomarkers in microvesicular RNA and cell-free DNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one of five biomarkers in microvesicular RNA and cell-free DNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

The present disclosure provides a method of treating Sjögren's syndrome in a subject, the method comprising: a) determining the expression level of at least one of five biomarkers in microvesicular RNA and cell-free DNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) administering at least one treatment to the subject when the expression level of the at least one biomarker is greater than or equal to its corresponding predetermined cutoff value.

The present disclosure provides a method of monitoring a Sjögren's syndrome treatment in a subject that has been administered the Sjögren's syndrome treatment, the method comprising: determining the expression level of at least one of five biomarkers in microvesicular RNA and cell-free DNA isolated from a saliva sample from the subject, wherein the five biomarkers comprise OAS1, OAS2, IRF5, IFIT1, and IFIT3; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; and b) identifying the presence of Sjögren's syndrome in the subject based on the expression level of the at least one biomarker.

The present disclosure provides a method of treating Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; and b) administering at least one treatment to the subject based on the expression level of the at least one biomarker.

The present disclosure provides a method of monitoring a Sjögren's syndrome treatment in a subject that has been administered the Sjögren's syndrome treatment, the method comprising: determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

The present disclosure provides a method of identifying the presence or absence of Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) comparing the expression level of the at least one biomarker to a corresponding predetermined cutoff value; c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

The present disclosure provides a method of treating Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) comparing the expression level of the at least one biomarker to a corresponding predetermined cutoff value; c) administering at least one treatment to the subject when the expression level of the at least one biomarker is greater than or equal to its corresponding predetermined cutoff value.

The present disclosure provides a method of identifying the presence or absence of Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) comparing the expression level of the at least one biomarker to a corresponding predetermined cutoff value; c) identifying the presence of Sjögren's syndrome in the subject when the expression level of the at least one biomarker is less than or equal to its corresponding predetermined cutoff value or identifying the absence of Sjögren's syndrome in the subject when the expression level of the at least one biomarker is greater than its corresponding predetermined cutoff value.

The present disclosure provides a method of treating Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) comparing the expression level of the at least one biomarker to a corresponding predetermined cutoff value; c) administering at least one treatment to the subject when the expression level of the at least one biomarker is less than or equal to its corresponding predetermined cutoff value.

The present disclosure provides a method of monitoring a Sjögren's syndrome treatment in a subject that has been administered the Sjögren's syndrome treatment, the method comprising: determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2, b) comparing the expression level of the at least one biomarker to a corresponding predetermined cutoff value; c) determining whether the patient is responding to the Sjögren's syndrome treatment based on whether the expression level of the at least one biomarker is greater than, equal to, or less than its corresponding predetermined cutoff value.

In some aspects of the preceding methods, step (a) can comprise determining the expression level of at least two of the seven biomarkers, or at least three of the seven biomarkers, or at least four of the seven biomarkers, at least five of the seven biomarkers, at least six of the seven biomarkers, or each of the seven biomarkers.

In some aspects, the at least one biomarker can be IFIT3.

In some aspects, the at least one biomarker can be OAS1.

In some aspects, the at least one biomarker can be OAS2.

In some aspects, the at least one biomarker can be IFIT1.

In some aspects, the at least one biomarker can be TMEM60.

In some aspects, the at least one biomarker can be ZNF395.

In some aspects, the at least one biomarker can be CPNE2.

The present disclosure provides a method of identifying the presence or absence of Sjögren's syndrome in a subject, the method comprising: a) determining the expression level of IFIT3 in microvesicular RNA isolated from a saliva sample from the subject; b) comparing the expression level of IFIT3 to a corresponding predetermined cutoff value; c) identifying the presence of Sjögren's syndrome in the subject when the expression level of IFIT3 is greater than or equal to its corresponding predetermined cutoff value or identifying the absence of Sjögren's syndrome in the subject when the expression level of IFIT3 is less than its corresponding predetermined cutoff value.

The present disclosure provides a method of treating Sjögren's syndrome in a subject, the method comprising: a) determining the expression level of IFIT3 in microvesicular RNA isolated from a saliva sample from the subject; b) comparing the expression level IFIT3 to a corresponding predetermined cutoff value; c) administering at least one treatment to the subject when the expression level of IFIT3 is greater than or equal to its corresponding predetermined cutoff value.

The present disclosure provides a method of identifying the presence or absence of Sjögren's syndrome in a subject, the method comprising: a) determining the expression level of OAS1 in microvesicular RNA isolated from a saliva sample from the subject; b) comparing the expression level of OAS1 to a corresponding predetermined cutoff value; c) identifying the presence of Sjögren's syndrome in the subject when the expression level of OAS1 is greater than or equal to its corresponding predetermined cutoff value or identifying the absence of Sjögren's syndrome in the subject when the expression level of OAS1 is less than its corresponding predetermined cutoff value.

The present disclosure provides a method of treating Sjögren's syndrome in a subject, the method comprising: a) determining the expression level of OAS1 in microvesicular RNA isolated from a saliva sample from the subject; b) comparing the expression level OAS1 to a corresponding predetermined cutoff value; c) administering at least one treatment to the subject when the expression level of OAS1 is greater than or equal to its corresponding predetermined cutoff value.

The present disclosure provides a method of identifying the presence or absence of Sjögren's syndrome in a subject, the method comprising: a) determining the expression level of OAS2 in microvesicular RNA isolated from a saliva sample from the subject; b) comparing the expression level of OAS2 to a corresponding predetermined cutoff value; c) identifying the presence of Sjögren's syndrome in the subject when the expression level of OAS2 is greater than or equal to its corresponding predetermined cutoff value or identifying the absence of Sjögren's syndrome in the subject when the expression level of OAS2 is less than its corresponding predetermined cutoff value.

The present disclosure provides a method of treating Sjögren's syndrome in a subject, the method comprising: a) determining the expression level of OAS2 in microvesicular RNA isolated from a saliva sample from the subject; b) comparing the expression level OAS2 to a corresponding predetermined cutoff value; c) administering at least one treatment to the subject when the expression level of OAS2 is greater than or equal to its corresponding predetermined cutoff value.

The present disclosure provides a method of identifying the presence or absence of Sjögren's syndrome in a subject, the method comprising: a) determining the expression level of IFIT1 in microvesicular RNA isolated from a saliva sample from the subject; b) comparing the expression level of IFIT1 to a corresponding predetermined cutoff value; c) identifying the presence of Sjögren's syndrome in the subject when the expression level of IFIT1 is greater than or equal to its corresponding predetermined cutoff value or identifying the absence of Sjögren's syndrome in the subject when the expression level of IFIT1 is less than its corresponding predetermined cutoff value.

The present disclosure provides a method of treating Sjögren's syndrome in a subject, the method comprising: a) determining the expression level of IFIT1 in microvesicular RNA isolated from a saliva sample from the subject; b) comparing the expression level IFIT1 to a corresponding predetermined cutoff value; c) administering at least one treatment to the subject when the expression level of IFIT1 is greater than or equal to its corresponding predetermined cutoff value.

The present disclosure provides a method of identifying the presence or absence of Sjögren's syndrome in a subject, the method comprising: a) determining the expression level of TMEM60 in microvesicular RNA isolated from a saliva sample from the subject; b) comparing the expression level of TMEM60 to a corresponding predetermined cutoff value; c) identifying the presence of Sjögren's syndrome in the subject when the expression level of TMEM60 is greater than or equal to its corresponding predetermined cutoff value or identifying the absence of Sjögren's syndrome in the subject when the expression level of TMEM60 is less than its corresponding predetermined cutoff value.

The present disclosure provides a method of treating Sjögren's syndrome in a subject, the method comprising: a) determining the expression level of TMEM60 in microvesicular RNA isolated from a saliva sample from the subject; b) comparing the expression level TMEM60 to a corresponding predetermined cutoff value: c) administering at least one treatment to the subject when the expression level of TMEM60 is greater than or equal to its corresponding predetermined cutoff value.

The present disclosure provides a method of identifying the presence or absence of Sjögren's syndrome in a subject, the method comprising: a) determining the expression level of CPNE2 in microvesicular RNA isolated from a saliva sample from the subject, b) comparing the expression level of CPNE2 to a corresponding predetermined cutoff value; c) identifying the presence of Sjögren's syndrome in the subject when the expression level of CPNE2 is greater than or equal to its corresponding predetermined cutoff value or identifying the absence of Sjögren's syndrome in the subject when the expression level of CPNE2 is less than its corresponding predetermined cutoff value.

The present disclosure provides a method of treating Sjögren's syndrome in a subject, the method comprising: a) determining the expression level of CPNE2 in microvesicular RNA isolated from a saliva sample from the subject; b) comparing the expression level CPNE2 to a corresponding predetermined cutoff value; c) administering at least one treatment to the subject when the expression level of CPNE2 is greater than or equal to its corresponding predetermined cutoff value.

The present disclosure provides a method of identifying the presence or absence of Sjögren's syndrome in a subject, the method comprising: a) determining the expression level of ZNF395 in microvesicular RNA isolated from a saliva sample from the subject; b) comparing the expression level of ZNF395 to a corresponding predetermined cutoff value; c) identifying the presence of Sjögren's syndrome in the subject when the expression level of ZNF395 is less than or equal to its corresponding predetermined cutoff value or identifying the absence of Sjögren's syndrome in the subject when the expression level of ZNF395 is greater than its corresponding predetermined cutoff value.

The present disclosure provides a method of treating Sjögren's syndrome in a subject, the method comprising: a) determining the expression level of ZNF395 in microvesicular RNA isolated from a saliva sample from the subject; b) comparing the expression level ZNF395 to a corresponding predetermined cutoff value; c) administering at least one treatment to the subject when the expression level of ZNF395 is less than or equal to its corresponding predetermined cutoff value.

The present disclosure provides a method of identifying the presence or absence of Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) identifying the presence or absence of Sjögren's syndrome based on the score. In some aspects, identifying the presence or absence of Sjögren's syndrome based on the score can comprise comparing the score to a predetermined cutoff value and determining the presence or absence of Sjögren's syndrome in the subject based on the relationship between the score and the predetermined cutoff value (e.g. is the score greater than the predetermined cutoff value, less than the predetermined cutoff value, or equal to the predetermined cutoff value).

The present disclosure provides a method of monitoring a Sjögren's syndrome treatment in a subject that has been administered the Sjögren's syndrome treatment, the method comprising: a) determining the expression level of at least one biomarker in microvesicular RNA isolated from a saliva sample from the subject; b) inputting the expression levels from step (a) into an algorithm to generate a score; c) determining whether the subject is responding to the Sjögren's syndrome treatment based on the score. In some aspects, determining whether the subject is responding to the Sjögren's syndrome treatment based on the score can comprise comparing the score to a predetermined cutoff value and determining if the subject is responding based on the relationship between the score and the predetermined cutoff value (e.g. is the score greater than the predetermined cutoff value, less than the predetermined cutoff value, or equal to the predetermined cutoff value).

The present disclosure provides a method of treating Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) administering at least one treatment to the subject based on the score. In some aspects, administering at least one treatment to the subject based on the score can further comprise comparing the score to a predetermined cutoff value and determining if the treatment is needed based on the relationship between the score and the predetermined cutoff value (e.g. is the score greater than the predetermined cutoff value, less than the predetermined cutoff value, or equal to the predetermined cutoff value).

The present disclosure provides a method of identifying the presence or absence of Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of treating Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) comparing the score to a predetermined cutoff value; d) administering at least one treatment to the subject when the score is greater than or equal to the predetermined cutoff value.

The present disclosure provides a method of identifying the presence or absence of Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method of treating Sjögren's syndrome in a subject, the method comprising: a) determining the expression level of at least one biomarker in microvesicular RNA isolated from a saliva sample from the subject; b) inputting the expression levels from step (a) into an algorithm to generate a score; c) comparing the score to a predetermined cutoff value; d) administering at least one treatment to the subject when the score is less than or equal to the predetermined cutoff value.

The present disclosure provides a method of monitoring a Sjögren's syndrome treatment in a subject that has been administered the Sjögren's syndrome treatment, the method comprising: a) determining the expression level of at least one biomarker in microvesicular RNA isolated from a saliva sample from the subject; b) inputting the expression levels from step (a) into an algorithm to generate a score; c) determining whether the patient is responding to the Sjögren's syndrome treatment based on whether the score is greater than, equal to, or less than the predetermined cutoff level.

The present disclosure provides a method of identifying the presence or absence of Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) identifying the presence or absence of Sjögren's syndrome in the subject based on the score.

The present disclosure provides a method of treating Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score, c) administering at least one treatment to the subject based on the score.

The present disclosure provides a method of identifying the presence or absence of Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of treating Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) administering at least one treatment to the subject when the score is greater than or equal to the predetermined cutoff value.

The present disclosure provides a method of identifying the presence or absence of Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method of treating Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) administering at least one treatment to the subject when the score is less than or equal to the predetermined cutoff value.

The present disclosure provides a method of monitoring a Sjögren's syndrome treatment in a subject that has been administered the Sjögren's syndrome treatment, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) determining whether the patient is responding to the Sjögren's syndrome treatment based on whether the score is greater than, equal to, or less than the predetermined cutoff level.

The present disclosure provides a method of identifying the presence or absence of Sjögren's syndrome in a subject, the method comprising: a) determining the expression level of at least one biomarker in microvesicular RNA and cell-free DNA isolated from a saliva sample from the subject; b) inputting the expression levels from step (a) into an algorithm to generate a score; c) identifying the presence or absence of Sjögren's syndrome based on the score. In some aspects, identifying the presence or absence of Sjögren's syndrome based on the score can comprise comparing the score to a predetermined cutoff value and determining the presence or absence of Sjögren's syndrome in the subject based on the relationship between the score and the predetermined cutoff value (e.g. is the score greater than the predetermined cutoff value, less than the predetermined cutoff value, or equal to the predetermined cutoff value).

The present disclosure provides a method of monitoring a Sjögren's syndrome treatment in a subject that has been administered the Sjögren's syndrome treatment, the method comprising: a) determining the expression level of at least one biomarker in microvesicular RNA and cell-free DNA isolated from a saliva sample from the subject; b) inputting the expression levels from step (a) into an algorithm to generate a score; c) determining whether the subject is responding to the Sjögren's syndrome treatment based on the score. In some aspects, determining whether the subject is responding to the Sjögren's syndrome treatment based on the score can comprise comparing the score to a predetermined cutoff value and determining if the subject is responding based on the relationship between the score and the predetermined cutoff value (e.g. is the score greater than the predetermined cutoff value, less than the predetermined cutoff value, or equal to the predetermined cutoff value).

The present disclosure provides a method of treating Sjögren's syndrome in a subject, the method comprising: a) determining the expression level of at least one biomarker in microvesicular RNA and cell-free DNA isolated from a saliva sample from the subject; b) inputting the expression levels from step (a) into an algorithm to generate a score; c) administering at least one treatment to the subject based on the score. In some aspects, administering at least one treatment to the subject based on the score can further comprise comparing the score to a predetermined cutoff value and determining if the treatment is needed based on the relationship between the score and the predetermined cutoff value (e.g. is the score greater than the predetermined cutoff value, less than the predetermined cutoff value, or equal to the predetermined cutoff value).

The present disclosure provides a method of identifying the presence or absence of Sjögren's syndrome in a subject, the method comprising: a) determining the expression level of at least one biomarker in microvesicular RNA and cell-free DNA isolated from a saliva sample from the subject; b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of treating Sjögren's syndrome in a subject, the method comprising: a) determining the expression level of at least one biomarker in microvesicular RNA and cell-free DNA isolated from a saliva sample from the subject; b) inputting the expression levels from step (a) into an algorithm to generate a score; c) comparing the score to a predetermined cutoff value; d) administering at least one treatment to the subject when the score is greater than or equal to the predetermined cutoff value.

The present disclosure provides a method of identifying the presence or absence of Sjögren's syndrome in a subject, the method comprising: a) determining the expression level of at least one biomarker in microvesicular RNA and cell-free DNA isolated from a saliva sample from the subject; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method of treating Sjögren's syndrome in a subject, the method comprising: a) determining the expression level of at least one biomarker in microvesicular RNA and cell-free DNA isolated from a saliva sample from the subject; b) inputting the expression levels from step (a) into an algorithm to generate a score; c) comparing the score to a predetermined cutoff value; d) administering at least one treatment to the subject when the score is less than or equal to the predetermined cutoff value.

The present disclosure provides a method of monitoring a Sjögren's syndrome treatment in a subject that has been administered the Sjögren's syndrome treatment, the method comprising: a) determining the expression level of at least one biomarker in microvesicular RNA and cell-free DNA isolated from a saliva sample from the subject; b) inputting the expression levels from step (a) into an algorithm to generate a score; c) determining whether the patient is responding to the Sjögren's syndrome treatment based on whether the score is greater than, equal to, or less than the predetermined cutoff level.

The present disclosure provides a method of identifying the presence or absence of Sjögren's syndrome in a subject, the method comprising: a) determining the expression level of at least one of 37 biomarkers in microvesicular RNA and cell-free DNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) identifying the presence or absence of Sjögren's syndrome in the subject based on the score.

The present disclosure provides a method of treating Sjögren's syndrome in a subject, the method comprising: a) determining the expression level of at least one of 37 biomarkers in microvesicular RNA and cell-free DNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) administering at least one treatment to the subject based on the score.

The present disclosure provides a method of identifying the presence or absence of Sjögren's syndrome in a subject, the method comprising: a) determining the expression level of at least one of 37 biomarker in microvesicular RNA and cell-free DNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of treating Sjögren's syndrome in a subject, the method comprising: a) determining the expression level of at least one of 37 biomarker in microvesicular RNA and cell-free DNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) administering at least one treatment to the subject when the score is greater than or equal to the predetermined cutoff value.

The present disclosure provides a method of identifying the presence or absence of Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method treating Sjögren's syndrome in a subject, the method comprising: a) determining the expression level of at least one of 37 biomarkers in microvesicular RNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) comparing the score to a predetermined cutoff value; d) administering at least one treatment to the subject when the score is less than or equal to the predetermined cutoff value.

The present disclosure provides a method of monitoring a Sjögren's syndrome treatment in a subject that has been administered the Sjögren's syndrome treatment, the method comprising: a) determining the expression level of at least one of 37 biomarker in microvesicular RNA and cell-free DNA isolated from a saliva sample from the subject, wherein the 37 biomarkers comprise ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395 and ZSCAN9; b) inputting the expression levels from step (a) into an algorithm to generate a score; c) determining whether the patient is responding to the Sjögren's syndrome treatment based on whether the score is greater than, equal to, or less than the predetermined cutoff level.

In some aspects of the preceding methods, step (a) can comprise determining the expression level of at least two of the 37 biomarkers, or at least three of the 37 biomarkers, or at least four of the 37 biomarkers, or at least five of the 37 biomarkers, or at least six of the 37 biomarkers, or at least seven of the 37 biomarkers, or at least eight of the 37 biomarkers, or at least nine of the 37 biomarkers, or at least 10 of the 37 biomarkers, or at least 11 of the 37 biomarkers, or at least 12 of the 37 biomarkers, or at least 13 of the 37 biomarkers, or at least four of the 14 biomarkers, or at least 15 of the 37 biomarkers, or at least 16 of the 37 biomarkers, or at least 17 of the 37 biomarkers, or at least 18 of the 37 biomarkers, or at least 19 of the 37 biomarkers, or at least 20 of the 37 biomarkers, or at least 21 of the 37 biomarkers, or at least 22 of the 37 biomarkers, or at least 23 of the 37 biomarkers, or at least 24 of the 37 biomarkers, or at least 25 of the 37 biomarkers, or at least 26 of the 37 biomarkers, or at least 27 of the 37 biomarkers, or at least 28 of the 37 biomarkers, or at least 29 of the 37 biomarkers, or at least 30 of the 37 biomarkers, or at least 31 of the 37 biomarkers, or at least 32 of the 37 biomarkers, or at least 33 of the 37 biomarkers, or at least 34 of the 37 biomarkers, or at least 35 of the 37 biomarkers, or at least 36 of the 37 biomarkers, or each of the 37 biomarkers.

The present disclosure provides a method of identifying the presence or absence of Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) identifying the presence or absence of Sjögren's syndrome in the subject based on the score.

The present disclosure provides a method of treating Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) administering at least one treatment to the subject based on the score.

The present disclosure provides a method of identifying the presence or absence of Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of treating Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) administering at least one treatment to the subject when the score is greater than or equal to the predetermined cutoff value.

The present disclosure provides a method of identifying the presence or absence of Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method of treating Sjögren's syndrome in a subject, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) administering at least one treatment to the subject when the score is less than or equal to the predetermined cutoff value.

The present disclosure provides a method of monitoring a Sjögren's syndrome treatment in a subject that has been administered the Sjögren's syndrome treatment, the method comprising: a) determining the expression level of at least one of seven biomarkers in microvesicular RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) determining whether the patient is responding to the Sjögren's syndrome treatment based on whether the score is greater than, equal to, or less than the predetermined cutoff level.

The present disclosure provides a method of identifying the presence or absence of Sjögren's syndrome in a subject, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) identifying the presence or absence of Sjögren's syndrome in the subject based on the score.

The present disclosure provides a method of treating Sjögren's syndrome in a subject, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) administering at least one treatment to the subject based on the score.

The present disclosure provides a method of identifying the presence or absence of Sjögren's syndrome in a subject, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) identifying the presence of Sjögren's syndrome in the subject when the score is greater than or equal to the predetermined cutoff value or identifying the absence of Sjögren's syndrome in the subject when score is less than its corresponding predetermined cutoff value.

The present disclosure provides a method of treating Sjögren's syndrome in a subject, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) administering at least one treatment to the subject when the score is greater than or equal to the predetermined cutoff value.

The present disclosure provides a method of identifying the presence or absence of Sjögren's syndrome in a subject, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) identifying the presence of Sjögren's syndrome in the subject when the score is less than or equal to the predetermined cutoff value or identifying the absence of Sjögren's syndrome in the subject when score is greater than its corresponding predetermined cutoff value.

The present disclosure provides a method of treating Sjögren's syndrome in a subject, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) comparing the score to a predetermined cutoff value; d) administering at least one treatment to the subject when the score is less than or equal to the predetermined cutoff value.

The present disclosure provides a method of monitoring a Sjögren's syndrome treatment in a subject that has been administered the Sjögren's syndrome treatment, the method comprising: a) determining the expression level of at least one of seven biomarkers in microvesicular RNA and cell-free RNA isolated from a saliva sample from the subject, wherein the seven biomarkers comprise IFIT3, OAS1, OAS2, IFIT1, TMEM60, ZNF395 and CPNE2; b) inputting the expression levels from step (a) into an algorithm to generate a score; c) determining whether the patient is responding to the Sjögren's syndrome treatment based on whether the score is greater than, equal to, or less than the predetermined cutoff level.

In some aspects of the preceding methods, step (a) can comprise determining the expression level of at least two of the seven biomarkers, or at least three of the seven biomarkers, or at least four of the seven biomarkers, or at least five of the seven biomarkers, or at least six of the seven biomarkers, or each of the seven biomarkers.

The present disclosure provides a method of identifying the presence or absence of Sjögren's syndrome in a subject, the method comprising: a) determining the expression level of at least one upregulated biomarker and the expression level of at least one downregulated biomarker in microvesicular RNA isolated from a saliva sample from the subject; b) comparing the expression level of each of the biomarkers to a corresponding predetermined cutoff value for each biomarker; and c) identifying the presence of Sjögren's syndrome in the subject when the expression level of the at least one upregulated biomarker is greater than or equal to its corresponding predetermined cutoff value and the expression level of the at least one downregulated biomarker is less than or equal to its corresponding predetermined cutoff value or identifying the absence of Sjögren's syndrome in the subject when the expression level of the at least one upregulated biomarker is less than its corresponding predetermined cutoff value and the expression level of the at least one downregulated biomarker is greater than its corresponding predetermined cutoff value.

The present disclosure provides a method of treating Sjögren's syndrome in a subject, the method comprising: a) determining the expression level of at least one upregulated biomarker and the expression level of at least one downregulated biomarker in microvesicular RNA isolated from a saliva sample from the subject; b) comparing the expression level of each of the biomarkers to a corresponding predetermined cutoff value for each biomarker; and c) administering at least one treatment to the subject when the expression level of the at least one upregulated biomarker is greater than or equal to its corresponding predetermined cutoff value and the expression level of the at least one downregulated biomarker is less than or equal to its corresponding predetermined cutoff value.

The present disclosure provides a method of identifying the presence or absence of Sjögren's syndrome in a subject, the method comprising: a) determining the expression level of at least one upregulated biomarker and the expression level of at least one downregulated biomarker in microvesicular RNA and cell-free DNA isolated from a saliva sample from the subject; b) comparing the expression level of each of the biomarkers to a corresponding predetermined cutoff value for each biomarker; and c) identifying the presence of Sjögren's syndrome in the subject when the expression level of the at least one upregulated biomarker is greater than or equal to its corresponding predetermined cutoff value and the expression level of the at least one downregulated biomarker is less than or equal to its corresponding predetermined cutoff value or identifying the absence of Sjögren's syndrome in the subject when the expression level of the at least one upregulated biomarker is less than its corresponding predetermined cutoff value and the expression level of the at least one downregulated biomarker is greater than its corresponding predetermined cutoff value.

The present disclosure provides a method of treating Sjögren's syndrome in a subject, the method comprising: a) determining the expression level of at least one upregulated biomarker and the expression level of at least one downregulated biomarker in microvesicular RNA and cell-free DNA isolated from a saliva sample from the subject; b) comparing the expression level of each of the biomarkers to a corresponding predetermined cutoff value for each biomarker; and c) administering at least one treatment to the subject when the expression level of the at least one upregulated biomarker is greater than or equal to its corresponding predetermined cutoff value and the expression level of the at least one downregulated biomarker is less than or equal to its corresponding predetermined cutoff value.

In some aspects, an upregulated biomarker can be selected from IFIT3, OAS1, OAS2, IFIT1, TMEM60 and CPNE2.

In some aspects, a downregulated biomarker can be ZNF395.

### General Methods and Definitions

Any of the following general methods and definitions can be applied to any of the gene signatures and/or classifiers described above.

In some aspects of the present disclosure, a biomarker can be an mRNA. In some aspects of the present disclosure, a biomarker can be a long intervening/intergenic non-coding RNA (lincRNA).

In some aspects, any method of the present disclosure can further comprise administering at least one treatment to a subject identified as having Sjögren's syndrome.

In some aspects, any method of the present disclosure, prior to step (a), can further comprise: i) isolating a plurality of microvesicles from a saliva sample from the subject and ii) extracting at least one microvesicular RNA from the plurality of isolated microvesicles.

In some aspects, any method of the present disclosure, prior to step (a), can further comprise: i) isolating a microvesicle fraction from a saliva sample from the subject, wherein the microvesicle fraction comprises a plurality of microvesicles and cfDNA: ii) extracting at least one microvesicular RNA and at least one cfDNA molecule from the isolated microvesicle fraction.

In some aspects of the methods of the present disclosure, isolating a plurality of microvesicles from a biological sample from the subject can comprise a processing step to remove cells, cellular debris or a combination of cells and cellular debris. A processing step can comprise filtering the sample, centrifuging the sample, or a combination of filtering the sample and centrifuging the sample. Centrifuging can comprise centrifuging at about 2000×g Filtering can comprise filtering the sample through a filter with a pore size of about 0.8 microns

In some aspects of the methods of the present disclosure, isolating a plurality of microvesicles can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating a microvesicle fraction, wherein the microvesicle fraction comprises a plurality of microvesicles and cfDNA can comprise ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

In some aspects of the methods of the present disclosure, isolating an at least one microvesicle is from a saliva sample can comprise contacting the saliva sample with at least one affinity agent that binds to at least one surface marker present on the surface the at least one microvesicle.

In some aspects, the microvesicular RNA can be isolated from a saliva sample using an extraction-free method. In some aspects, the extraction-free method comprises direct lysis of microvesicles in the saliva sample without prior isolation of the microvesicles to yield microvesicular RNA. Without wishing to be bound by theory, the extraction-free method that does not include a microvesicle isolation step is more easily adapted to automated methods, particular for high-throughput sample processing. In some aspects, an extraction-free method can comprise directly adding a lysis solution to a saliva sample.

Other microvesicle and microvesicle fraction isolation procedures are described in US 2017-0088898 A1, US 2016-0348095 A1, US 2016-0237422 A1, US 2015-0353920 A1, U.S. Pat. No. 10,465,183 and US 2019-0284548 A1, the contents of each of which are incorporated herein by reference in their entireties. The methods of the present disclosure can comprise any of the methods described in the aforementioned United States patent Publications and United States patents.

Other microvesicle and microvesicle fraction isolation procedures are described in WO 2018/076018, the contents of which are incorporated herein by reference in their entireties. The methods of the present disclosure can comprise any of the methods described in the aforementioned PCT Application Publication.

In some aspects of the methods of the present disclosure, determining the expression level of a biomarker can comprise quantitative PCR (qPCR), quantitative real-time PCR, semi-quantitative real-time PCR, digital PCR (dPCR), reverse transcription PCR (RT-PCR), reverse transcription quantitative PCR (qRT-PCR), microarray analysis, sequencing, next-generation sequencing (NGS), high-throughput sequencing, direct-analysis or any combination thereof.

In some aspects of the methods of the present disclosure, determining the expression level of a biomarker can comprise quantitative PCR (qPCR), quantitative real-time PCR, semi-quantitative real-time PCR, reverse transcription PCR (RT-PCR), reverse transcription quantitative PCR (qRT-PCR), microarray analysis, sequencing, next-generation sequencing (NGS), high-throughput sequencing, direct-analysis, droplet digital PCR, or any combination thereof.

In some aspects of the methods of the present disclosure, an expression level of a biomarker or endogenous control gene can correspond to a cycle threshold (Ct) value when the expression level is determined using quantitative PCR (qPCR), quantitative real-time PCR, semi-quantitative real-time PCR, reverse transcription PCR (RT-PCR) or reverse transcription quantitative PCR (qRT-PCR).

In some aspects, any of the expression levels of a biomarker can be normalized using methods known in the art. For example, expression levels of biomarkers measured in the methods disclosed herein can be normalized to the expression level of an endogenous control gene and/or a reference biomarker. In some aspects, normalizing the expression level of a biomarker to the expression level of an endogenous control gene and/or a reference biomarker can comprise subtracting the expression level of the endogenous control gene and/or a reference biomarker from the expression level of the biomarker. Accordingly, in aspects wherein the expression levels are measured as Ct values, the normalized expression value of a biomarker can be the Ct value of the biomarker minus the Ct value of the endogenous control gene and/or a reference biomarker. In some aspects, normalizing the expression level of a biomarker to the expression level of an endogenous control gene can comprise dividing the expression level of the biomarker by the expression level of the endogenous control gene and/or a reference biomarker.

In some aspects of the methods of the present disclosure wherein determining the expression level of a biomarker comprises sequencing, next-generation sequencing (NGS), high-throughput sequencing, or any combination thereof, at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, or any combination thereof can correspond to subject's transcriptome.

In some aspects of the methods of the present disclosure, microvesicular RNA and/or cell-free DNA that has been extracted from a plurality of isolated microvesicles and/or an isolated microvesicle fraction, and/or isolated from an extraction-free process can be subjected to library preparation procedures that are known in the art for the preparation of a library for sequencing, including next-generation sequencing and/or high-throughput sequencing.

In some aspects, microvesicular RNA and/or cfDNA isolated from a plurality of isolated microvesicles or a microvesicle fraction may be further processed in one or more steps, as described herein. These one or more steps can be performed concurrently or in any order.

Extracted microvesicular RNA can be further processed by fragmentation. Extracted cfDNA can be further processed by fragmentation.

Extracted microvesicular RNA can be further processed to selectively remove ribosomal DNA and/or RNA sequences from the extracted microvesicular RNA. In some aspects, selectively removing ribosomal DNA and/or RNA sequences can comprise the use of enzymatic reagents, including, but not limited to, RNase H or any other restriction enzyme. In some aspects, selectively removing ribosomal DNA and/or RNA sequences can comprise contacting the extracted microvesicular RNA with at least one affinity agent that binds to the ribosomal DNA and/or sequences. In some aspects, selectively removing ribosomal DNA and/or RNA sequences can comprise: i) contacting the extracted microvesicular RNA with biotinylated probes that hybridize to ribosomal DNA and/or RNA sequences; and ii) removing the hybridized probes using streptavidin conjugated paramagnetic beads.

Extracted cfDNA can be further processed to selectively remove ribosomal DNA and/or RNA sequences from the extracted cfDNA. In some aspects, selectively removing ribosomal DNA and/or RNA sequences can comprise the use of enzymatic reagents, including, but not limited to, RNase H or any other restriction enzyme. In some aspects, selectively removing ribosomal DNA and/or RNA sequences can comprise contacting the extracted cfDNA with at least one affinity agent that binds to the ribosomal DNA and/or sequences. In some aspects, selectively removing ribosomal DNA and/or RNA sequences can comprise: i) contacting the extracted cfDNA with biotinylated probes that hybridize to ribosomal DNA and/or RNA sequences; and ii) removing the hybridized probes using streptavidin conjugated paramagnetic beads.

Extracted microvesicular RNA can be further processed to reverse transcribe the extracted microvesicular RNA into cDNA. Reverse transcription can be performed using methods known in the art.

cDNA and/or cfDNA can be further processed to construct a double-stranded DNA sequencing library from the reverse transcribed cDNA and/or cfDNA. The double-stranded DNA sequencing library can be further amplified prior to sequencing. The amplification can be selective amplification of at least one biomarker. Selective amplification can be performed by PCR, wherein the PCR comprises the use of PCR primers that selectively hybridize to the at least one biomarker. A double-stranded DNA sequencing library or an amplified double-stranded DNA sequencing library can further comprise selectively enriching for at least one biomarker from the double-stranded DNA sequencing library or the amplified double-stranded DNA sequencing library. Selectively enriching at least one biomarker from the double-stranded DNA sequencing library or the amplified double-stranded DNA sequencing library can comprise the use of hybrid capture methods known in the art. Hybrid capture methods can comprise contacting the double-stranded DNA sequencing library or the amplified double-stranded DNA sequencing library with at least one affinity agent that binds to the at least one biomarker to be enriched. In a non-limiting example, selectively enriching at least one biomarker from the double-stranded DNA sequencing library or the amplified double-stranded DNA sequencing library can comprise: i) contacting the double-stranded DNA sequencing library or the amplified double-stranded DNA sequencing library with at least one biotinylated probe that binds to the at least one biomarker; and ii) enriching the hybridized probes using streptavidin conjugated paramagnetic beads.

cDNA can be further processed to amplify the cDNA. The amplification can be selective amplification of at least one biomarker. Selective amplification can be performed by PCR, wherein the PCR comprises the use of PCR primers that selectively hybridize to the at least one biomarker. cDNA or amplified cDNA can be further processed to selectively enrich at least one biomarker. Selectively enriching at least one biomarker from cDNA or amplified cDNA can comprise the use of hybrid capture methods known in the art. The hybrid capture methods can comprise contacting the cDNA or amplified cDNA with at least one affinity agent that binds to the at least one biomarker to be enriched. In a non-limiting example, selectively enriching at least one biomarker from cDNA or amplified cDNA can comprise: i) contacting the cDNA or amplified cDNA with at least one biotinylated probe that binds to the at least one biomarker; and ii) enriching the hybridized probes using streptavidin conjugated paramagnetic beads.

cfDNA can be further processed to amplify the cfDNA. The amplification can be selective amplification of at least one biomarker. Selective amplification can be performed by PCR, wherein the PCR comprises the use of PCR primers that selectively hybridize to the at least one biomarker. cfDNA or amplified cfDNA can be further processed to selectively enrich at least one biomarker. Selectively enriching at least one biomarker from cfDNA or amplified cfDNA can comprise the use of hybrid capture methods known in the art. The hybrid capture methods can comprise contacting the cfDNA or amplified cfDNA with at least one affinity agent that binds to the at least one biomarker to be enriched. In a non-limiting example, selectively enriching at least one biomarker from cfDNA or amplified cfDNA can comprise: i) contacting the cfDNA or amplified cfDNA with at least one biotinylated probe that binds to the at least one biomarker; and ii) enriching the hybridized probes using streptavidin conjugated paramagnetic beads.

Extracted microvesicular RNA can be further processed to amplify the extracted microvesicular RNA. The amplification can be selective amplification of at least one biomarker. Selective amplification can be performed by PCR, wherein the PCR comprises the use of PCR primers that selectively hybridize to the at least one biomarker. Extracted microvesicular RNA or amplified microvesicular RNA can be further processed to selectively enrich at least one biomarker. Selectively enriching at least one biomarker from extracted microvesicular RNA or amplified microvesicular RNA can comprise the use of hybrid capture methods known in the art. The hybrid capture methods can comprise contacting the extracted microvesicular RNA or amplified microvesicular RNA with at least one affinity agent that binds to the at least one biomarker to be enriched. In a non-limiting example, selectively enriching at least one biomarker from extracted microvesicular RNA or amplified microvesicular RNA can comprise: i) contacting the extracted microvesicular RNA or amplified microvesicular RNA with at least one biotinylated probe that binds to the at least one biomarker; and ii) enriching the hybridized probes using streptavidin conjugated paramagnetic beads.

In some aspects, determining the expression level of at least one biomarker in microvesicular RNA, or in a mixture of microvesicular RNA and cfDNA can comprise fragmenting the microvesicular RNA.

In some aspects, determining the expression level of at least one biomarker in microvesicular RNA, or in a mixture of microvesicular RNA and cfDNA, can comprise reverse-transcribing the microvesicular RNA into cDNA. In some aspects, the cDNA can be amplified. The amplification can be a selective amplification of at least one biomarker. In some aspects, cfDNA or amplified cfDNA can be further processed to selectively enrich at least one biomarker. Selectively enriching at least one biomarker from cfDNA or amplified cfDNA can comprise the use of hybrid capture methods known in the art. The hybrid capture methods can comprise contacting the cfDNA or amplified cfDNA with at least one affinity agent that binds to the at least one biomarker to be enriched. In a non-limiting example, selectively enriching at least one biomarker from cfDNA or amplified cfDNA can comprise: i) contacting the cfDNA or amplified cfDNA with at least one biotinylated probe that binds to the at least one biomarker; and ii) enriching the hybridized probes using streptavidin conjugated paramagnetic beads. In some aspects, following the selective enrichment of at least one biomarker, the enriched at least one biomarker can be amplified. The amplification can be a selective amplification of the at least one biomarker.

In some aspects, the cDNA or amplified cDNA can be used to construct a double-stranded DNA sequencing library using techniques known in the art. cDNA or amplified cDNA that has been selectively enriched for at least one biomarker can be used to construct a double-stranded DNA sequencing library. cDNA or amplified cDNA that has been selectively enriched for at least one biomarker and then amplified again can be used to construct a double-stranded DNA sequencing library. Constructing a double-stranded DNA sequencing can be performed using methods known in the art.

In some aspects, determining the expression level of at least one biomarker in cfDNA, or in a mixture of microvesicular RNA and cfDNA, can comprise fragmenting the cfDNA.

In some aspects, determining the expression level of at least one biomarker in microvesicular RNA, or in a mixture of microvesicular RNA and cfDNA, can comprise amplifying the cfDNA. The amplification can be a selective amplification of at least one biomarker. In some aspects, cfDNA or amplified cfDNA can be further processed to selectively enrich at least one biomarker. Selectively enriching at least one biomarker from cfDNA or amplified cfDNA can comprise the use of hybrid capture methods known in the art. The hybrid capture methods can comprise contacting the cfDNA or amplified cfDNA with at least one affinity agent that binds to the at least one biomarker to be enriched. In a non-limiting example, selectively enriching at least one biomarker from cfDNA or amplified cfDNA can comprise: i) contacting the cfDNA or amplified cfDNA with at least one biotinylated probe that binds to the at least one biomarker; and ii) enriching the hybridized probes using streptavidin conjugated paramagnetic beads. In some aspects, following the selective enrichment of at least one biomarker, the enriched at least one biomarker can be amplified. The amplification can be a selective amplification of the at least one biomarker.

In some aspects, the cfDNA or amplified cfDNA can be used to construct a double-stranded DNA sequencing library using techniques known in the art. cfDNA or amplified cfDNA that has been selectively enriched for at least one biomarker can be used to construct a double-stranded DNA sequencing library. cfDNA or amplified cfDNA that has been selectively enriched for at least one biomarker and then amplified again can be used to construct a double-stranded DNA sequencing library. Constructing a double-stranded DNA sequencing can be performed using methods known in the art.

In some aspects of the methods of the present disclosure, the hybrid-capture methods described herein substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome. In some aspects of the methods of the present disclosure, the hybrid-capture methods described herein result in a significant depletion in microbial nucleic acids.

In some aspects, any of the methods described herein can be performed in an automation-compatible instrument. Automation-compatible instruments include, but are not limited to, Tecan liquid handling device, a Hamilton liquid handling device, or any other platforms capable of performing high-throughput specimen processing in a research or diagnostic setting.

The present disclosure provides a method of purifying nucleic acid transcripts that correspond to the human transcriptome from a saliva sample from a human subject, the method comprising: a) isolating a plurality of microvesicles from the saliva sample; b) extracting microvesicular RNA from the plurality of isolated microvesicles; c) purifying nucleic acid transcripts that correspond to the human transcriptome from the extracted microvesicular RNA by performing hybrid-capture, wherein the product of the hybrid-capture is substantially enriched for nucleic acid transcripts that correspond to the human transcriptome and is substantially depleted of nucleic acids that are derived from microbes. In some aspects, the product of the hybrid-capture can comprise at least about 70%, or at least about 75%, or at least about 80%, or at least about 85%, or at least about 90%, or at least about 95%, or at least about 99%, or at least about 99.5% nucleic acid transcripts that correspond to the human transcriptome. In some aspects, the product of the hybrid-capture can comprise no more than about 25%, or about 20%, or about 15%, or about 10%, or about 5%, or about 2.5%, or about 1%, or about 0.5% nucleic acid transcripts that are derived from a microbe.

The present disclosure provides a method of purifying nucleic acid transcripts that correspond to the human transcriptome from a saliva sample from a human subject, the method comprising: a) isolating a plurality of microvesicles and cfDNA from the saliva sample; b) extracting microvesicular RNA from the plurality of isolated microvesicles; c) purifying nucleic acid transcripts that correspond to the human transcriptome from the extracted microvesicular RNA and isolated cfDNA by performing hybrid-capture, wherein the product of the hybrid-capture is substantially enriched for nucleic acid transcripts that correspond to the human transcriptome and is substantially depleted of nucleic acids that are derived from microbes. In some aspects, the product of the hybrid-capture can comprise at least about 70%, or at least about 75%, or at least about 80%, or at least about 85%, or at least about 90%, or at least about 95%, or at least about 99%, or at least about 99.5% nucleic acid transcripts that correspond to the human transcriptome. In some aspects, the product of the hybrid-capture can comprise no more than about 25%, or about 20%, or about 15%, or about 10%, or about 5%, or about 2.5%, or about 1%, or about 0.5% nucleic acid transcripts that are derived from a microbe.

In some aspects, the subject is human.

In some aspects, the subject can have been previously diagnosed with Sjögren's syndrome based on the presence of anti-Ro autoantibody (also referred to as Anti-Sjögren's syndrome-related antigen A autoantibodies [anti-SSA]) in at least one biological sample from the subject.

In some aspects, the subject can have been previously identified as anti-Ro autoantibody negative based on the absence of the anti-Ro autoantibody in at least one biological sample from the subject.

In some aspects, the subject can have been previously diagnosed with Sjögren's syndrome based on the presence of anti-La autoantibody (also referred to as Anti-Sjögren's syndrome-related antigen B autoantibodies [anti-SSB]) in at least one biological sample from the subject.

In some aspects, the subject can have been previously identified as anti-La autoantibody negative based on the absence of the anti-La autoantibody in at least one biological sample from the subject.

In some aspects of the methods of the present disclosure, a predetermined cutoff value can be selected to have a negative predictive value (NPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected to have a positive predictive value (PPV) of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected to have a sensitivity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, a predetermined cutoff value can be selected to have a specificity of at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%, or at least about 99.9%.

In some aspects of the methods of the present disclosure, an algorithm can be the product of a feature selection wrapper algorithm. In some aspects of the methods of the present disclosure, an algorithm can be the product of a machine learning algorithm. In some aspects of the methods of the present disclosure, an algorithm can be the product of a trained classifier built from at least one predictive classification algorithm. In some aspects of the methods of the present disclosure, an algorithm can be the product of a of a logistic regression model. A logistic regression model can comprise LASSO regularization.

In some aspects of the methods of the present disclosure a predictive classification algorithm, a feature selection wrapper algorithm, and/or a machine learning algorithm can comprise XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta (see Kursa M B, Rudnicki W R. Feature Selection with the Boruta Package. J Stat Softw 2010; 36 (11), incorporated herein by reference in its entirety) or any combination thereof.

In some aspects of the methods of the present disclosure, a predetermined cutoff value can be calculated using at least one receiver operating characteristic (ROC) curve. In some aspects of the methods of the present disclosure, a predetermined cutoff value can be calculated and/or selected to have any of the features described herein (e.g., a specific sensitivity, specificity, PPV, NPV or any combination thereof) using any method known in the art, as would be appreciated by the skilled artisan.

In some aspects of the methods of the present disclosure, an algorithm can a product of a feature selection wrapper algorithm, machine learning algorithm, trained classifier, logistic regression model or any combination thereof, that was trained to identify Sjögren's syndrome in a subject using: a) the expression levels of the at least one, or the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15, or the at least 16, or the at least 17, or the at least 18, or the at least 19, or the at least 20, or the at least 21, or the at least 22, or the at least 23, or the at least 24, or the at least 25, or the at least 26, or the at least 27, or the at least 28, or the at least 29, or the at least 30, or the at least 31, or the at least 32, or the at least 33, or the at least 34, or the at least 35, or the at least 36, or the at least 37 biomarkers in at least one biological sample from at least one subject who does not have Sjögren's syndrome; and b) the expression levels of the at least one, or the at least two, or the at least three, or the at least four, or the at least five, or the at least six, or the at least seven, or the at least eight, or the at least nine, or the at least 10, or the at least 11, or the at least 12, or the at least 13, or the at least 14, or the at least 15, or the at least 16, or the at least 17, or the at least 18, or the at least 19, or the at least 20, or the at least 21, or the at least 22, or the at least 23, or the at least 24, or the at least 25, or the at least 26, or the at least 27, or the at least 28, or the at least 29, or the at least 30, or the at least 31, or the at least 32, or the at least 33, or the at least 34, or the at least 35, or the at least 36, or the at least 37 biomarkers in at least one biological sample from at least one subject who has Sjögren's syndrome.

In some aspects, a predetermined cutoff value can be the expression level of a biomarker in a biological sample collected from a subject who does not have Sjögren's syndrome. In some aspects, a predetermine cutoff value can be the mean (average) expression level of a biomarker from a plurality of samples collected from a plurality of subjects who do not have Sjögren's syndrome.

In some aspects, a predetermined cutoff value can be the expression level of a biomarker in a biological sample collected from a subject who has Sjögren's syndrome. In some aspects, a predetermine cutoff value can be the mean (average) expression level of a biomarker from a plurality of samples collected from a plurality of subjects who have Sjögren's syndrome.

A treatment can comprise at least one therapeutically effective amount of an artificial tear, cevimeline (Evoxac®) pilocarpine (Salagen®), a supersaturated calcium phosphate rinse (e.g. NeutraSal®), cyclosporine (including ophthalmic emulsions, e.g. Restasis® and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof.

A treatment can comprise at least one therapeutically effective amount of a UCB5857 targeting PI3Kδ by selectively inhibiting PI3Kδ preventing transmission of cell surface receptor signaling; CFZ533 targeting CD40 by being Fc silent antibody to CD40 preventing B cell stimulation and differentiation without depletion; AMG557 targeting ICOS by inhibiting activation of TFH; VAY736 targeting BAFF-R by being an antibody to BAFF-R preventing BAFF-mediated B cell proliferation and survival; Low-dose IL-2 targeting CD4⁺CD25⁺ T cells by expanding Treg cells; Rituximab+belimumab targeting CD20 B cells and BAFF by eliciting anti-CD20-dependent depletion of B cells combined with BAFF blockade to decrease survival of self-reactive B cells; Tocilizumab targeting IL-6R by causing blockade of IL-6R preventing IL-6-dependent TH17 and TFH cell differentiation; Abatacept targeting CD80/86 through CTLA4-Ig binding of CD80/86 prevents co-stimulation-dependent activation of CD4 T cells; RSLV-132, a fully human biologic Fc fusion protein, targeting extracellular RNA to prevent activation of the immune system via Toll-like receptors and the interferon pathway; and VIB4920, a fusion protein binding to CD40L on activated T cells, blocking their interaction with CD40-expressing B cells.

A treatment can comprise surgery. A surgery can comprise a surgery to seal the tear ducts that drain tears from the subject's eyes (also referred to as a punctal occlusion). The tear ducts may be sealed, for example, by inserting collagen or silicone plugs into the ducts.

In some aspects of the methods of the present disclosure, a saliva sample can be collected at the subject's home through the use of a sample home-collection device.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

### EXEMPLARY EMBODIMENTS

Embodiment 1. A method of identifying the presence or absence of Sjögren's syndrome in a subject, the method comprising:
a) determining the expression level of at least one biomarker in microvesicular RNA isolated from a saliva sample from the subject;
b) comparing the expression level of the at least one biomarker to a corresponding predetermined cutoff value;
c) identifying the presence of Sjögren's syndrome in the subject when the expression level of the at least one biomarker is greater than or equal to its corresponding predetermined cutoff value or identifying the absence of Sjögren's syndrome in the subject when the expression level of the at least one biomarker is less than its corresponding predetermined cutoff value.

Embodiment 2. A method of monitoring a Sjögren's syndrome treatment in a subject, wherein the subject has been administered the Sjögren's syndrome treatment, the method comprising:
a) determining the expression level of at least one biomarker in microvesicular RNA isolated from a saliva sample from the subject;
b) comparing the expression level of the at least one biomarker to a corresponding predetermined cutoff value;
c) determining whether the patient is responding to the Sjögren's syndrome treatment based on whether the expression level of the at least one biomarker is greater than, equal to, or less than its corresponding predetermined cutoff value.

Embodiment 3. A method of identifying the presence or absence of Sjögren's syndrome in a subject, the method comprising:
a) determining the expression level of at least one biomarker in microvesicular RNA isolated from a saliva sample from the subject;
b) inputting the expression levels from step (a) into an algorithm to generate a score;
c) comparing the score to a predetermined cutoff value;
d) identifying the presence of Sjögren's syndrome in the subject when the score is greater than or equal to the predetermined cutoff value or identifying the absence of Sjögren's syndrome in the subject when score is less than its corresponding predetermined cutoff value.

Embodiment 4. A method of identifying the presence or absence of Sjögren's syndrome in a subject, the method comprising:
a) determining the expression level of at least one biomarker in microvesicular RNA isolated from a saliva sample from the subject;
b) inputting the expression levels from step (a) into an algorithm to generate a score;
c) comparing the score to a predetermined cutoff value;
d) identifying the presence of Sjögren's syndrome in the subject when the score is less than or equal to the predetermined cutoff value or identifying the absence of Sjögren's syndrome in the subject when score is greater than its corresponding predetermined cutoff value.

Embodiment 5. A method of monitoring a Sjögren's syndrome treatment in a subject, wherein the subject has been administered the Sjögren's syndrome treatment, the method comprising:
a) determining the expression level of at least one biomarker in microvesicular RNA isolated from a saliva sample from the subject;
b) inputting the expression levels from step (a) into an algorithm to generate a score;
c) comparing the score to a predetermined cutoff value;
d) determining whether the patient is responding to the Sjögren's syndrome treatment based on whether the score is greater than, equal to, or less than the predetermined cutoff value.

Embodiment 6. The method of any of the preceding embodiments, further comprising prior to step (a):
i) isolating a plurality of microvesicles from the saliva sample from the subject; and
ii) extracting at least one microvesicular RNA from the plurality of isolated microvesicles.

Embodiment 7. The method of any of the preceding embodiments, wherein isolating a plurality of microvesicles from a biological sample from the subject comprises a processing step to remove cells, cellular debris or a combination of cells and cellular debris.

Embodiment 8. The method of any of the preceding embodiments, wherein the processing step comprises filtering the sample, centrifuging the sample, or a combination of filtering the sample and centrifuging the sample.

Embodiment 9. The method of any of the preceding embodiments, wherein centrifuging comprises centrifuging at about 2000×g.

Embodiment 10 The method of any of the preceding embodiments, wherein filtering comprises filtering the sample through a filter with a pore size of about 0.8 microns.

Embodiment 11. The method of any of the preceding embodiments, wherein isolating a plurality of microvesicles comprises ultrafiltration, ultracentrifugation, ion-exchange chromatography, size exclusion chromatography, density gradient centrifugation, centrifugation, differential centrifugation, immunoabsorbent capture, affinity purification, affinity exclusion, microfluidic separation, nanomembrane concentration or any combination thereof.

Embodiment 12. The method of any of the preceding embodiments, wherein the at least one microvesicle is isolated from the saliva sample by contacting the saliva sample with at least one affinity agent that binds to at least one surface marker present on the surface the at least one microvesicle.

Embodiment 13. The method of any of the preceding embodiments, wherein step (a) further comprises:
(i) determining the expression level of at least one reference biomarker;
(ii) normalizing the expression level of the at least one biomarker to the expression level of the at least one reference biomarker.

Embodiment 14. The method of any of the preceding embodiments, wherein inputting the expression levels from step (a) into an algorithm to generate a score comprises inputting the normalized expression levels from step (a) into an algorithm to generate a score.

Embodiment 15. The method of any of the preceding embodiments, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

Embodiment 16. The method of preceding embodiments, wherein determining the expression level of a biomarker comprises sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

Embodiment 17. The method of any of the preceding embodiments, wherein the predetermined cutoff value has a negative predictive value of at least about 80%, or at least about 85%, or at least about 90%, or at least about 95%, or at least about 99%, or at least about 99.9%.

Embodiment 18. The method of any of the preceding embodiments, wherein the predetermined cutoff value has a positive predictive value of at least about 80%, or at least about 85%, or at least about 90%, or at least about 95%, or at least about 99%, or at least about 99.9%.

Embodiment 19. The method of any of the preceding embodiments, wherein the predetermined cutoff value has a sensitivity of at least about 80%, or at least about 85%, or at least about 90%, or at least about 95%, or at least about 99%, or at least about 99.9%.

Embodiment 20. The method of any of the preceding embodiments, wherein the predetermined cutoff value has a specificity of at least about 80%, or at least about 85%, or at least about 90%, or at least about 95%, or at least about 99%, or at least about 99.9%.

Embodiment 21. The method of any of the preceding embodiments, wherein the algorithm is the product of a feature selection wrapper algorithm.

Embodiment 22. The method of any of the preceding embodiments, wherein the algorithm is the product of a machine learning algorithm.

Embodiment 23. The method of any of the preceding embodiments, wherein the algorithm is the product of a trained classifier built from at least one predictive classification algorithm.

Embodiment 24. The method of any of the preceding embodiments, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), cforest, classification and regression tree (CART), treebag, k nearest-neighbor (knn), neural network (nnet), support vector machine-radial (SVM-radial), support vector machine-linear (SVM-linear), naïve bayes (NB), multilayer perceptron (mlp) or any combination thereof.

Embodiment 25. The method of any of the preceding embodiments, wherein the algorithm is the product of a logistic regression model.

Embodiment 26. The method of any of the preceding embodiments, wherein the logistic regression model comprises a LASSO regularization.

Embodiment 27. The method of any of the preceding embodiments, wherein the predetermined cutoff value is calculated using at least one receiver operating characteristic (ROC) curve.

Embodiment 28. The method of any of the preceding embodiments, wherein measuring expression levels in step (a) further comprises selectively enriching for at least one biomarker.

Embodiment 29. The method of any of the preceding embodiments, wherein the at least one biomarker is selectively enriched by hybrid-capture.

Embodiment 30. The method of claim 28, wherein the hybrid-capture substantially enriches nucleic acid transcripts that correspond to the human transcriptome or any subset of the human transcriptome.

Embodiment 31. The method of claim 29, wherein the hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome.

Embodiment 32. The method of any one of embodiments 29-31, wherein the hybrid-capture results in a significant depletion in microbial nucleic acids.

Embodiment 33. The method of any of the preceding embodiments, further comprising administering at least one treatment to a subject identified as having Sjögren's syndrome.

Embodiment 34. The method of any of the preceding embodiments, wherein the at least one treatment comprises administering at least one therapeutically effective amount of an cevimeline (Evoxac®) pilocarpine (Salagen®), a supersaturated calcium phosphate rinse (e.g. NeutraSal®), cyclosporine (including ophthalmic emulsions, e.g. Restasis® and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof.

Embodiment 35. The method of any of the preceding embodiments, wherein the at least one treatment comprises surgery.

Embodiment 36. The method of any of the preceding embodiments, wherein the surgery comprises sealing the tear ducts of the subject.

Embodiment 37. The method of any of the preceding embodiments, wherein the saliva sample is collected at the subject's home through the use of a sample home-collection device.

### EXAMPLES

### Example 1

About 1 ml of saliva was collected from seven subjects with Sjögren's syndrome and seven healthy subjects who did not have Sjögren's syndrome. Microvesicles were isolated from the saliva samples using the methods of the present disclosure. Microvesicular RNA was extracted from the isolated microvesicles and the RNA was analyzed using next-generation sequencing. As part of the next-generation sequencing analysis, hybrid-capture was used to enrich for human exome transcripts and long intervening/intergenic noncoding RNAs (lincRNAs). ERCC RNA spike-in mix was also used as a control. Hybrid-capture was also used to enrich for human transcripts of any size of a defined panel of genes consisting of at least two genes, and in any combination with lincRNA and ERCC RNA as a control.

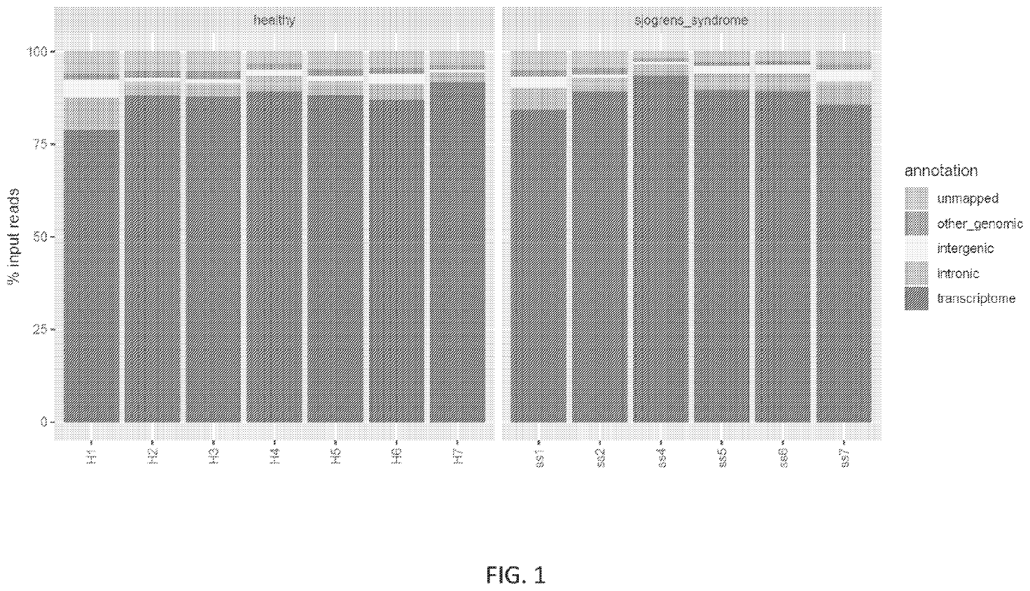

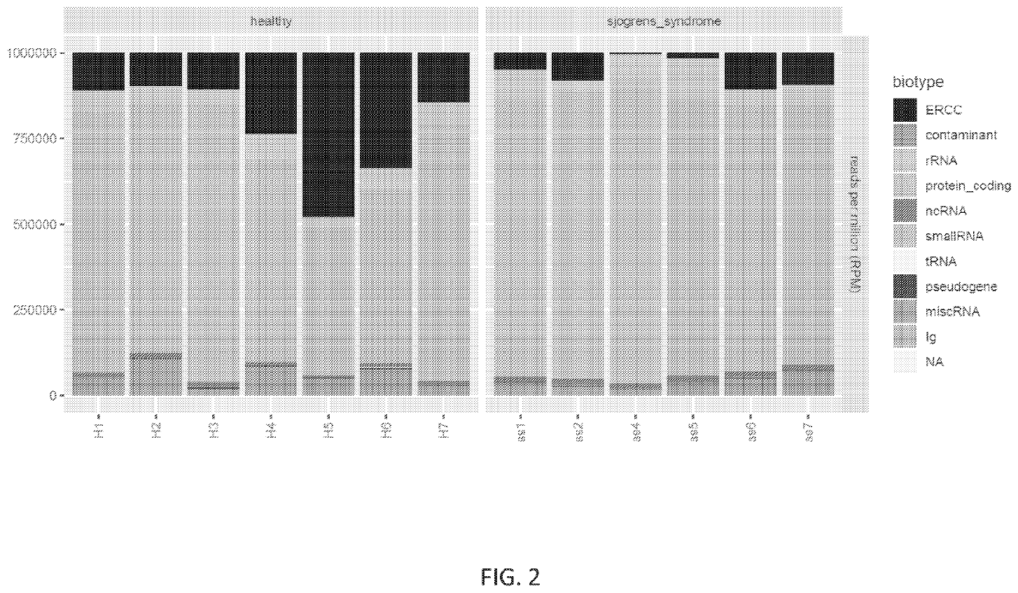

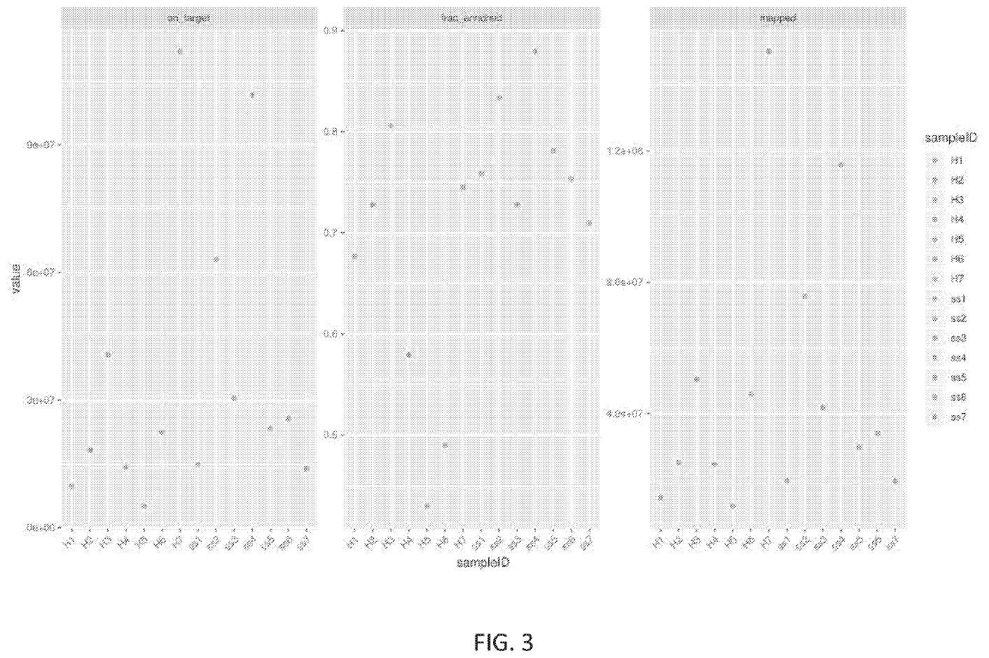

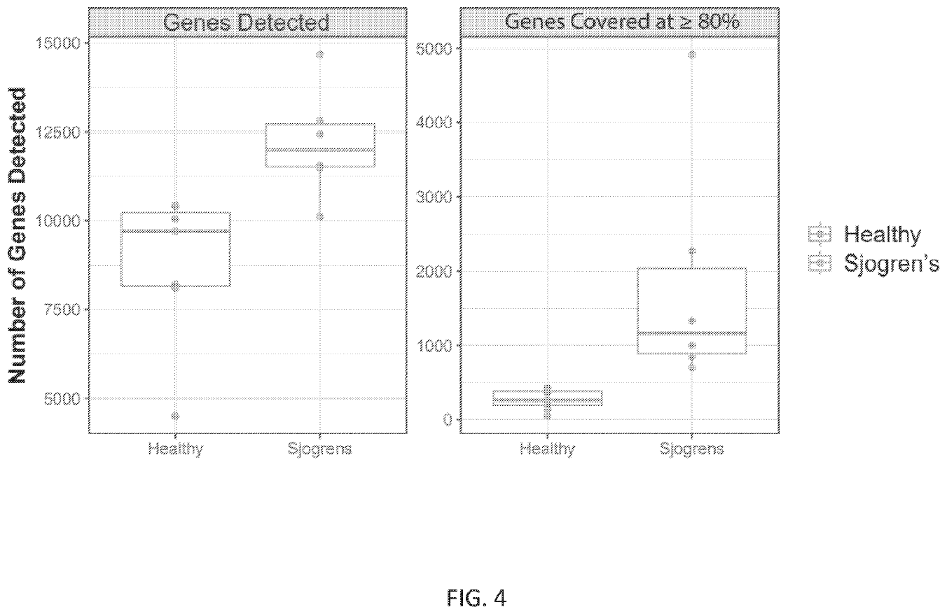

As shown in FIG. 1, over 80% of the sequencing reads from the analysis of the microvesicular RNA were mapped to the human transcriptome. This is in stark contrast with results from existing studies, which reported less than 20% of human reads in nucleic acids extracted from exosomal RNA isolated from saliva samples (e.g. Rozowsky et al., Cell Press, 8:352-357.e3., 2019; Yeri et al. Scientific Reports, 7, 44061, 2017). Thus, the methods of the present disclosure, for the first time, allow for the significant enrichment of human nucleic acid transcripts and the substantial depletion of microbial nucleic acids in exosomal nucleic acids isolated from saliva. As shown in FIG. 2, although sample-to sample variability was observed in the biotype of the different sequencing reads, the sequencing reads from most sample results in over 75% protein coding reads. As shown in FIG. 3, most samples had over 70% of on-target reads that corresponded with a transcript that was targeted for hybrid-capture. As shown in FIG. 4, at a sequencing depth of just 10 M reads, over 10,000 protein coding genes were detected from saliva exosomes. Significant proportion of these genes bad more than 80% gene body coverage.

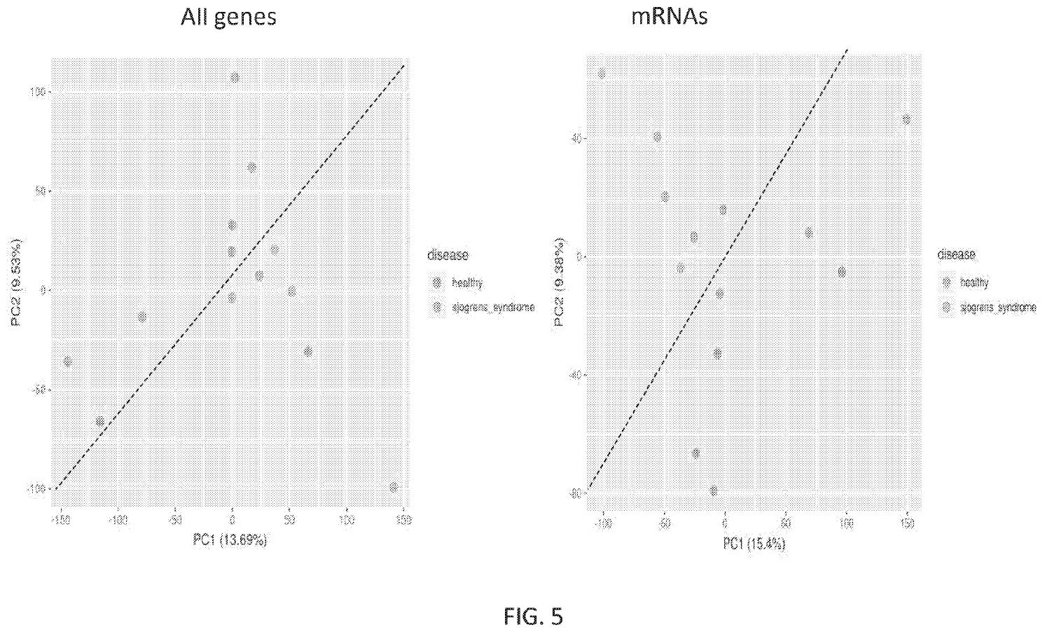

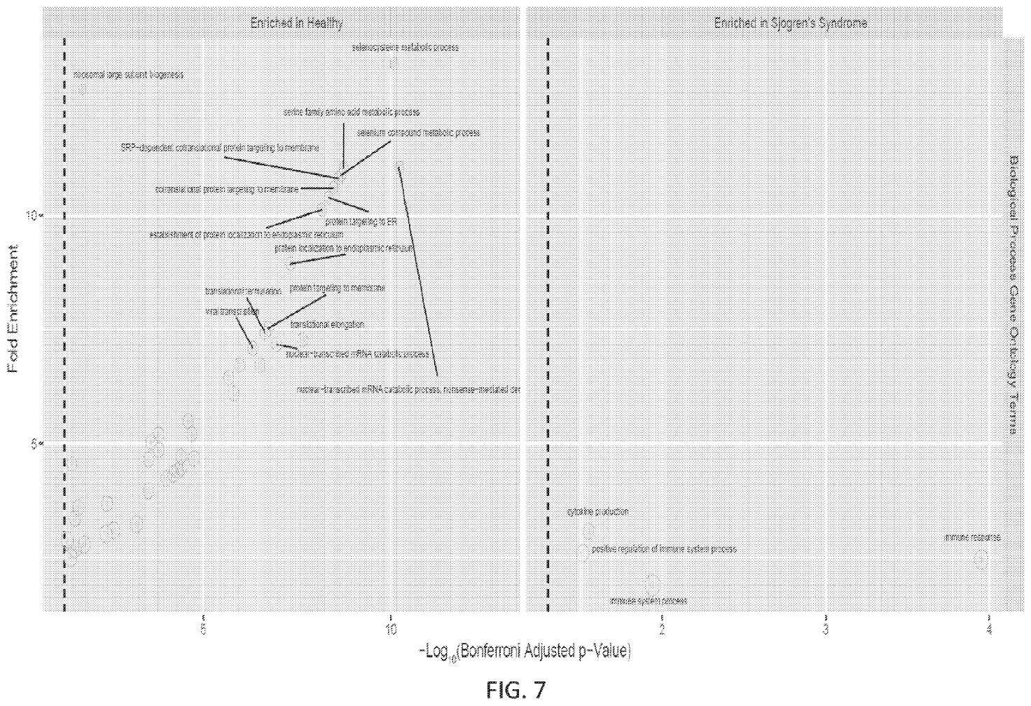

Differential expression analysis of the sequencing results from the Sjögren's syndrome patients and healthy patients showed that there were approximately 438 genes that were upregulated in Sjögren's syndrome samples. Of these 438 genes, 359 were mRNAs and 13 were lincRNAs. The differential expression analysis also showed that 198 genes were downregulated in Sjögren's syndrome samples. Of these 198 genes, 175 were mRNAs and 3 were lincRNAs. An enrichment in interferon alpha, interferon beta and interferon gamma response genes was observed in the Sjögren's syndrome samples. FIG. 7 shows a graph depicting certain groups of genes that were enriched in either Sjögren's syndrome samples or healthy samples. As shown in FIG. 5, principle component analysis using all genes or mRNAs differentiates between Sjögren's syndrome samples and healthy samples. As shown in Table 1, the upregulated genes in the Sjögren's syndrome samples included OAS1, OAS2, IRF5, IFIT1 and IFIT3.

TABLE 1

| | Transcripts per | | | |
|---|---|---|---|---|
| | million (TPM) | TPM | Fold | |
| | Sjögren's | Healthy | change | |
| Gene | syndrome Samples | Samples | (log2) | p-value |
| OAS1 | 121.9 | 2.2 | 5.69 | 0.0000524 |
| OAS2 | 45.4 | 3 | 3.93 | 0.0982142 |
| IRF5 | 3 | 0 | 8.48 | 0.0000021 |
| IFIT1 | 258.1 | 28 | 2.88 | 0.1660608 |
| IFIT3 | 412.2 | 52.7 | 2.78 | 0.0919973 |

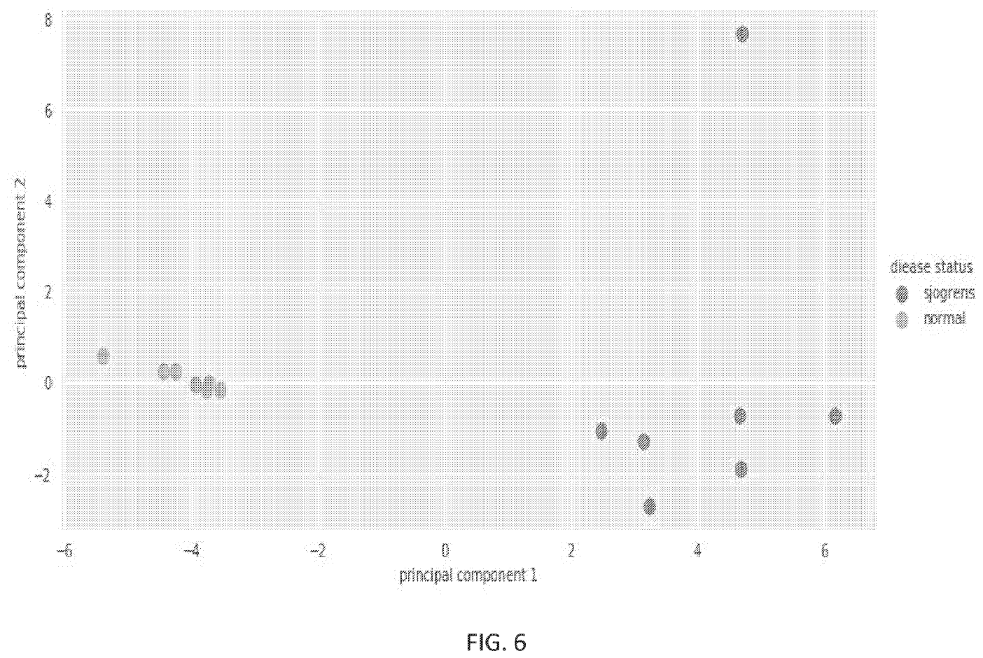

Feature selection was performed on the differentially expressed genes and 37 genes were identified: ABHD4, AQP1, ATF3, CDK7, CLEC5A, COG4, CPNE2, CYBB, ELOVL1, ETV7, GPR183, GTPBP4, HACE1, HIRA, ING1, IRF5, LAP3, MED30, MXD3, NDFIP1, PCED1B-AS1, RETSTAT, RFTN1, RPS14, SCAP, SEC23A, SEPT5, SIK1, SLC8B1, SLC04C1, SNTB2, TMEM60, TNFAIP8L2, U2AF1, UQCRC2, ZNF395, ZSCAN9. As shown in FIG. 6, principle component analysis using these 37 genes differentiates between Sjögren's syndrome samples and healthy samples.

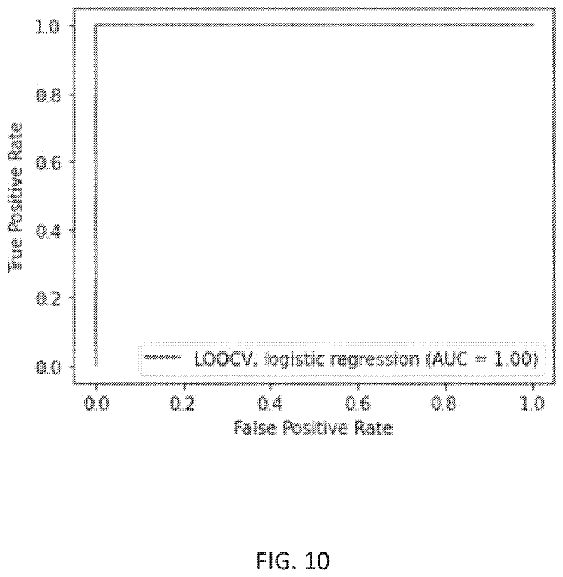

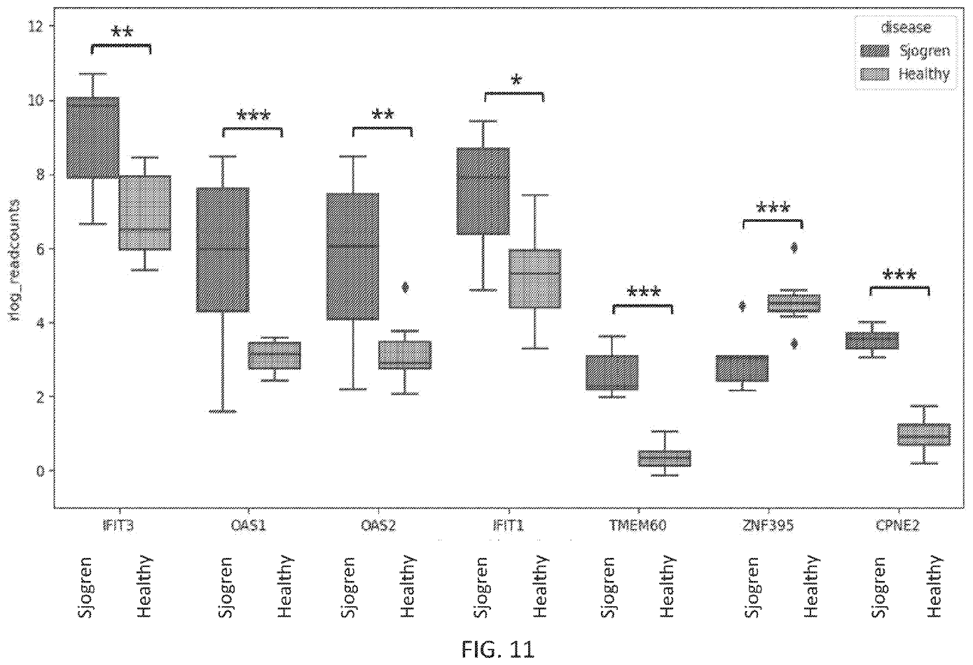

Further analysis of the five upregulated genes described above and the 37 genes identified by feature selection yielded a gene signature comprising seven genes: IFIT3, OAS1, OAS2, IFIT1, TMEM60, ZNF395, CPNE2. A gene signature comprising these seven genes was successfully identified the presence or absence of Sjögren's syndrome in subjects, as demonstrated in FIG. 10, which shows leave-one out cross-validation analysis of the gene signature. Further, the expression of each of the seven genes in healthy samples vs. Sjögren's syndrome samples was further analyzed, and the results are shown in FIG. 11. As shown in FIG. 11 and Tables 2 and 3, IFIT3, OAS1, OAS2, IFIT1, TMEM60 and CPNE2 were upregulated in Sjögren's syndrome samples as compared to healthy samples, while ZNF395 was downregulated in Sjögren's syndrome samples as compared to healthy samples.

TABLE 2

| | IFIT3 | OAS1 | OAS2 | IFIT1 | TMEM60 | ZNF395 | CPNE2 | Disease |
|---|---|---|---|---|---|---|---|---|
| Sample | (counts) | (counts) | (counts) | (counts) | (counts) | (counts) | (counts) | State |
| H11 | 7.860 | 3.387 | 3.180 | 5.320 | 0.049 | 4.555 | 1.725 | Healthy |
| H12 | 5.411 | 3.592 | 2.842 | 3.282 | 0.548 | 4.466 | 0.985 | Healthy |
| H13 | 8.009 | 2.639 | 2.673 | 4.805 | 1.060 | 4.503 | 1.460 | Healthy |
| H14 | 5.699 | 3.121 | 2.049 | 6.421 | 0.473 | 6.031 | 0.909 | Healthy |
| H15 | 8.427 | 3.498 | 4.942 | 7.423 | −0.141 | 4.861 | 0.196 | Healthy |
| SS1 | 9.852 | 7.275 | 7.320 | 8.360 | 2.834 | 3.071 | 3.299 | Sjogren |
| SS2 | 10.220 | 7.935 | 7.605 | 9.018 | 2.155 | 2.239 | 4.009 | Sjogren |
| SS3 | 6.638 | 3.469 | 2.705 | 4.863 | 2.272 | 4.437 | 3.544 | Sjogren |
| SS4 | 8.591 | 5.130 | 5.481 | 7.395 | 1.984 | 2.151 | 3.805 | Sjogren |
| SS5 | 10.715 | 8.468 | 8.472 | 9.417 | 3.604 | 2.579 | 3.282 | Sjogren |
| SS6 | 9.870 | 5.960 | 6.067 | 7.906 | 3.340 | 3.077 | 3.032 | Sjogren |
| SS7 | 7.218 | 1.591 | 2.172 | 5.353 | 2.178 | 3.023 | 3.586 | Sjogren |
| H9 | 6.487 | 2.429 | 3.760 | 3.956 | 0.339 | 4.157 | 0.754 | Healthy |
| H10 | 6.242 | 2.878 | 2.902 | 5.452 | 0.224 | 3.420 | 0.621 | Healthy |

TABLE 3

| geneID | log2FoldChange |
|---|---|
| TMEM60 | −10.51456568 |
| CPNE2 | −8.566494559 |
| OAS1 | −5.84259229 |
| ZNF395 | 3.65489249 |
| OAS2 | −3.873306618 |
| IFIT3 | −2.756644833 |
| IFIT1 | −2.834203342 |

The analysis presented in FIGS. 10 and 11, and Tables 2 and 3, demonstrate that these seven genes, both individually, and in various combinations, can be used to identify the presence or absence of Sjögren's syndrome in a subject.

Taken together, these results demonstrate that microvesicular RNA isolated from saliva samples can be used to differentiate between subjects having Sjögren's syndrome and subject that do not have Sjögren's syndrome.

### Example 2

About 1 ml of saliva was collected from seven subjects with Sjögren's syndrome and eleven healthy subjects who did not have Sjögren's syndrome. Microvesicles were isolated from the saliva samples using the methods of the present disclosure. Microvesicular RNA was extracted from the isolated microvesicles and the RNA was analyzed using next-generation sequencing. As part of the next-generation sequencing analysis, hybrid-capture was used to enrich for human exome transcripts and long intervening/intergenic noncoding RNAs (lincRNAs). ERCC RNA spike-in mix was also used as a control. Hybrid-capture was also used to enrich for human transcripts of any size of a defined panel of genes consisting of at least two genes as a subset of the human transcriptome, and in any combination with lincRNA and ERCC RNA as a control.

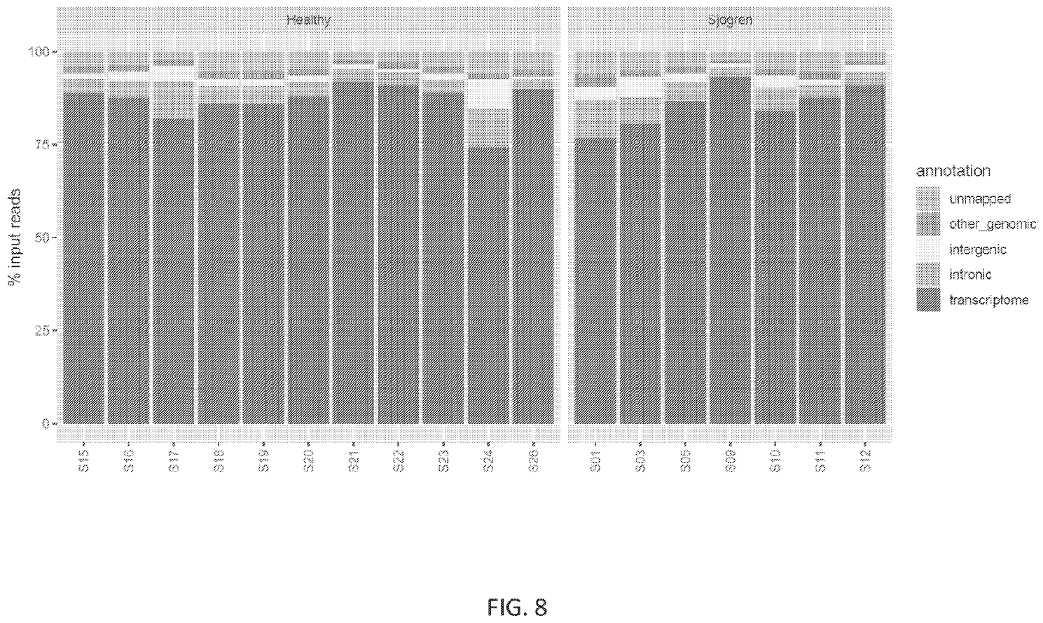

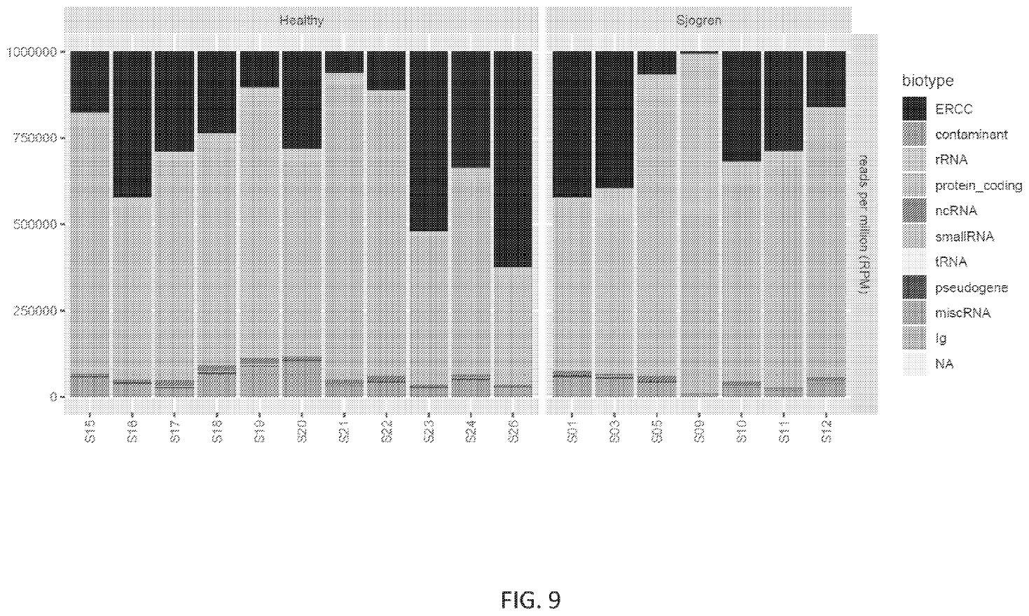

As shown in FIG. 8, in the majority of the samples, over 80% of the sequencing reads from the analysis of the microvesicular RNA were mapped to the human transcriptome. As shown in FIG. 9, although sample-to sample variability was observed in the biotype of the different sequencing reads, the sequencing reads from most sample results in protein coding reads. Thus, like Example 1, the methods of the present disclosure, for the first time, allow for the significant enrichment of human nucleic acid transcripts and the substantial depletion of microbial nucleic acids in exosomal nucleic acids isolated from saliva.

What is claimed is:

1. A method of identifying the presence or absence of Sjögren's syndrome in a subject, the method comprising:
a) determining the expression level of at least one of seven biomarkers in microvesicular RNA isolated from a saliva sample from the subject,
wherein the seven biomarkers comprise IFIT3, OAS1, OAS2, IFIT1, TMEM60, ZNF395 and CPNE2; and
b) identifying the presence of Sjögren's syndrome in the subject based on the expression level of the at least one biomarker.

2. The method of claim 1, wherein identifying the presence of Sjögren's syndrome in the subject based on the expression level of the at least one biomarker comprises:
i) inputting the expression levels from step (a) into an algorithm to generate a score;
ii) comparing the score to a predetermined cutoff value; and
iii) determining the presence or absence of Sjögren's syndrome in the subject based on comparison between the score and the predetermined cutoff value.

3. The method of claim 2, wherein the algorithm is the product of a feature selection wrapper algorithm, a machine learning algorithm, a trained classifier built from at least one predictive classification algorithm or any combination thereof.

4. The method of claim 3, wherein the predictive classification algorithm, the feature selection wrapper algorithm, and/or the machine learning algorithm comprises XGBoost (XGB), random forest (RF), Lasso and Elastic-Net Regularized Generalized Linear Models (glmnet), Linear Discriminant Analysis (LDA), cforest, classification and regression tree (CART), treebag, k nearest-neighbor (knn), neural network (nnet), support vector machine-radial (SVM-radial), support vector machine-linear (SVM-linear), naïve Bayes (NB), multilayer perceptron (mlp), Boruta or any combination thereof.

5. The method of claim 4, wherein the algorithm is the product of a trained classifier is trained to identify Sjögren's syndrome in a subject using:
i) the expression level of the at least one biomarker in at least one biological sample from at least one subject who does not have Sjögren's syndrome; and
ii) the expression levels of the at least one biomarker in at least one biological sample from at least one subject who has Sjögren's syndrome.

6. The method of claim 1, wherein:
i) the at least one biomarker is at least one of IFIT3, OAS1, OAS2, IFIT1, TMEM60 and CPNE2, wherein the presence of Sjögren's syndrome in the subject is identified when the expression level of the at least one biomarker is greater than or equal to a predetermined cutoff value; and/or
ii) the at least one biomarker is ZNF395, wherein the presence of Sjögren's syndrome in the subject is identified when the expression level of the at least one biomarker is less than or equal to a predetermined cutoff value

7. The method of any one of the preceding claims, wherein step (a) comprises determining the expression level of:
a) at least two of the seven biomarkers;
b) at least three of the seven biomarkers;
c) at least four of the seven biomarkers;
d) at least five of the seven biomarkers;
e) at least six of the seven biomarkers; or
f) each of the seven biomarkers.

8. The method of any of the preceding claims, further comprising prior to step (a):
i) isolating a plurality of microvesicles from the saliva sample from the subject; and
ii) extracting at least one microvesicular RNA from the plurality of isolated microvesicles.

9. The method of any of the preceding claims, wherein the at least one microvesicle is isolated from the saliva sample by contacting the saliva sample with at least one affinity agent that binds to at least one surface marker present on the surface the at least one microvesicle.

10. The method of any of the preceding claims, wherein step (a) further comprises:
(i) determining the expression level of at least one reference biomarker;
(ii) normalizing the expression level of the at least one biomarker to the expression level of the at least one reference biomarker.

11. The method of any of the preceding claims, wherein inputting the expression levels from step (a) into an algorithm to generate a score comprises inputting the normalized expression levels from step (a) into an algorithm to generate a score.

12. The method of any of the preceding claims, wherein determining the expression level of a biomarker comprises quantitative PCR (qPCR), quantitative real-time PCR, semi-quantitative real-time PCR, reverse transcription PCR (RT-PCR), reverse transcription quantitative PCR (qRT-PCR), digital PCR (dPCR), microarray analysis, sequencing, next-generation sequencing (NGS), high-throughput sequencing, direct-analysis or any combination thereof.

13. The method of claim 12, wherein determining the expression level of a biomarker comprises sequencing, next-generation sequencing (NGS), high-throughput sequencing or any combination thereof, wherein at least about 70%, or at least about 75%, or at least about 80%, or at least about 85%, or at least about 90%, or at least about 95%, or at least about 99%, or at least about 99.5% of the sequencing reads obtained by the sequencing, next-generation sequencing (NGS), high-throughput sequencing, direct-analysis or any combination thereof correspond to subject's transcriptome.

14. The method of any of the preceding claims, wherein the predetermined cutoff value has a negative predictive value of at least about 80%, or at least about 85%, or at least about 90%, or at least about 95%, or at least about 99%, or at least about 99.9%.

15. The method of any of the preceding claims, wherein the predetermined cutoff value has a positive predictive value of at least about 80%, or at least about 85%, or at least about 90%, or at least about 95%, or at least about 99%, or at least about 99.9%.

16. The method of any of the preceding claims, wherein the predetermined cutoff value has a sensitivity of at least about 80%, or at least about 85%, or at least about 90%, or at least about 95%, or at least about 99%, or at least about 99.9%.

17. The method of any of the preceding claims, wherein the predetermined cutoff value has a specificity of at least about 80%, or at least about 85%, or at least about 90%, or at least about 95%, or at least about 99%, or at least about 99.9%.

18. The method of any of the preceding claims, wherein the predetermined cutoff value is calculated using at least one receiver operating characteristic (ROC) curve.

19. The method of any of the preceding claims, wherein measuring expression levels in step (a) further comprises selectively enriching for at least one biomarker.

20. The method of any of the preceding claims, wherein the at least one biomarker is selectively enriched by hybrid-capture, preferably wherein:
i) the hybrid-capture substantially enriches nucleic acid transcripts that correspond to the human transcriptome such that at least about 70%, or at least about 75%, or at least about 80%, or at least about 85%, or at least about 90%, or at least about 95%, or at least about 99%, or at least about 99.5% of enriched nucleic acid transcripts correspond to the human transcriptome; and/or
ii) the hybrid-capture results in a significant depletion in microbial nucleic acids

21. The method of any of the preceding claims, further comprising administering at least one treatment to a subject identified as having Sjögren's syndrome.

22. The method of claim 23, wherein the at least one treatment comprises:
i) administering at least one therapeutically effective amount of an cevimeline (Evoxac®) pilocarpine (Salagen®), a supersaturated calcium phosphate rinse (e.g. NeutraSal®), cyclosporine (including ophthalmic emulsions, e.g. Restasis® and Cequa™), tacrolimus eye drops, abatacept (Orencia®), rituximab (Rituxan®), tocilizumab (Actemra®), hydroxypropyl cellulose (Lacrisert®), lifitegrast (including ophthalmic solutions, e.g. Xiidra®), LO2A eye drops, rebamipide eye drops, topical autologous serum, intravenous immunoglobulins, dexamethasone eye drops (Maxidex™), an immunosuppressive medication, a nonsteroidal anti-inflammatory medication, an arthritis medication, an antifungal medication, hydroxychloroquine (Plaquenil), methotrexate (Trexall), LOU064, INCB050465 or any combination thereof,
ii) surgery, preferably wherein the surgery comprises sealing the tear ducts of the subject; or
iii) a combination thereof.

23. The method of any of the preceding claims, wherein the saliva sample is collected at the subject's home through the use of a sample home-collection device.
